(19) United States
(12) Patent Application Publication
(10) Pub. No.: US 2018/0214295 A1
(43) Pub. Date: Aug. 2, 2018

(54) THERAPEUTIC FOOTWEAR WITH SUPPORT DEVICE

(71) Applicant: Osborn Medical Corp., Centennial, CO (US)

(72) Inventor: William D. Davis, Denver, CO (US)

(21) Appl. No.: 15/881,474

(22) Filed: Jan. 26, 2018

Related U.S. Application Data
(63) Continuation-in-part of application No. 29/592,246, filed on Jan. 27, 2017.
(60) Provisional application No. 62/451,463, filed on Jan. 27, 2017.

(51) Int. Cl. A61F 5/01; A61F 5/30

(52) U.S. Cl. CPC A61F 5/0113; A61F 5/30

(57) ABSTRACT

A therapeutic footwear apparatus includes a boot and a lateral support device for laterally supporting the boot. The boot includes a lower leg support portion and a foot support portion. The lower leg support portion is configured to at least partially wrap a lower leg. The lateral support device has a mounting face abutted to a side wall of the lower leg support portion. The mounting face has a curved surface complementary to a curved shape of the side wall of the lower leg support portion.

### CROSS-REFERENCE TO RELATED APPLICATION(S)

This application claims priority to U.S. Provisional Patent Application No. 62/451,463 filed on Jan. 27, 2017, titled THERAPEUTIC FOOTWEAR WITH SUPPORT DEVICE, and is a Continuation-in-Part of U.S. Design Patent application Ser. No. 29/592,246, filed on Jan. 27, 2017, titled SUPPORT DEVICE, the disclosures of which are hereby incorporated by reference in their entireties.

### BACKGROUND

Hospital patients are sometimes bed ridden for long periods of time. In some situations the patient can experience problems associated from lying down in one position for so long. For example, patients with vascular disease need to have good blood circulation to prevent clotting. The proper pre and post-operative footwear is needed to prevent vasoconstriction and promote vasodilation. In addition to maintaining proper blood circulation, the foot must be prevented from pointing in the downward direction or leaning to the side, a problem recognized in the industry that can lead to foot drop. Foot drop is a condition that may occur after lying in bed for some time without getting up or walking. Foot drop is the dropping of the forefoot due to weakness, damage to the peroneal nerve or paralysis of the muscles in the anterior portion of the lower leg. It is characterized by the inability or difficulty in moving the ankle and toes upward and thereby leading to the improper rotation of the foot. Heel or foot ulcers are another condition that can develop as a result of the heel rubbing against the pre or post-operative footwear or pressure being placed on the heel while lying in a hospital bed.

### SUMMARY

In general terms, this disclosure is directed to therapeutic footwear with a support device. In one possible configuration and by non-limiting example, the footwear includes a boot, and the support device is configured to laterally support the boot. Various aspects are described in this disclosure, which include, but are not limited to, the following aspects.

One aspect is a heel float therapeutic footwear apparatus including a boot and a lateral support device. The boot includes a lower leg support portion and a foot support portion. The lower leg support portion is configured to at least partially wrap a lower leg. The lower leg support portion has a front wall, a back wall, and opposite side walls. The front wall at least partially surrounds a front portion of the lower leg, the back wall at least partially surrounds a back portion of the lower leg, and the side walls at least partially surrounds sides of the lower leg. The foot support portion extends from the lower leg support portion. The lateral support device is configured to laterally support the lower leg support portion. The lateral support device has a mounting face abutted to one of the side walls. The mounting face has a curved surface complementary to a curved shape of the side walls.

Another aspect is a heel float therapeutic footwear apparatus including a heel float therapeutic boot and a lateral support device. The heel float therapeutic boot includes a lower leg support portion and a foot support portion. The lower leg support portion is configured to support a lower leg of a patient. The lower leg support portion includes a lower leg support cradle having a raised portion configured to elevate a heel of the patient when the patient is lying on the patient's back to reduce pressure on the patient's heel. The lower leg support portion has a front wall, a back wall, and opposite side walls. The front wall at least partially surrounds a front portion of the lower leg, the back wall at least partially surrounds a back portion of the lower leg, and the side walls at least partially surrounds sides of the lower leg. The foot support portion is connected to and extending from the lower leg support portion. The foot support portion includes a foot support platform extending from the lower leg support cradle. The lateral support device is detachably mounted to one of the side walls and configured to laterally support the lower leg support portion. The lateral support device has a mounting face abutted to the one of the side walls. The mounting face has a shape complementary to a shape of the side walls.

### BRIEF DESCRIPTION OF THE DRAWINGS

FIG. 1 is a perspective view of an example of a footwear apparatus.

FIG. 2 is a perspective view of an example support device.

FIG. 3 is a side view of the support device of FIG. 2.

FIG. 4 illustrates the support device in a first position.

FIG. 5 illustrates the support device in a second position.

FIG. 6 is perspective view of an example of a heel float therapeutic boot with a wing support structure fastened in accordance with the principles of the present disclosure.

FIG. 7 is perspective view of the heel float therapeutic boot shown in FIG. 6 with the wing support structure unfastened.

FIG. 8 is a perspective view of the heel float therapeutic boot shown in FIG. 7 with upper overlapping members unfastened.

FIG. 9 is a perspective back view of the heel float therapeutic boot shown in FIG. 6.

FIG. 10 is a component view of a portion of the heel float therapeutic boot shown in FIG. 8 taken along line 5-5.

FIG. 11 is a perspective view of an example of a heel float cradle in an upright position in accordance with the principles of the present disclosure.

FIG. 12 is a cross-sectional view of a portion of the heel float cradle shown in FIG. 11.

FIG. 13 is a perspective view of a portion of the heel float therapeutic boot shown in FIG. 7.

FIG. 14 is a bottom perspective view of the heel float therapeutic boot shown in FIG. 6.

FIG. 15 is a component view of a portion of the heel float therapeutic boot shown in FIG. 7 taken along line 10-10.

FIG. 16 is a perspective view of the heel float cradle shown in FIG. 11 without being in an upright position.

FIG. 17 is an enlarged view of a portion of the heel float cradle shown in FIG. 16.

FIG. 18 is a cross-sectional view of the heel float therapeutic boot shown in FIG. 6 with a lower leg of a patient.

FIG. 19 is a side perspective view of the heel float therapeutic boot shown in FIG. 6.

FIG. 20 is a bottom perspective view of the heel float therapeutic boot shown in FIG. 6 with the wing support structure unfastened.

FIG. 21 is an enlarged view of the wing support structure shown in FIG. 20.

FIG. 22 is a flow chart illustrating a method of making a heel float therapeutic footwear apparatus in accordance with the principles of the present disclosure.

FIG. 23 is a perspective view of another example of the heel float therapeutic boot.

FIG. 24 is a front view of the heel float therapeutic boot of FIG. 23.

FIG. 25 is a first side view (e.g., a left side view) of the heel float therapeutic boot of FIG. 23.

FIG. 26 is a second side view (e.g., a right side view) of the heel float therapeutic boot of FIG. 23.

FIG. 27 is a rear view of the heel float therapeutic boot of FIG. 23.

FIG. 28 is a right side view of the heel float therapeutic boot without a foot support device.

FIG. 29 is a left side view of the heel float therapeutic boot without the foot support device.

FIG. 30 is a bottom view of the heel float therapeutic boot of FIG. 23.

FIG. 31 is a perspective view of the heel float therapeutic boot shown in FIG. 23 with an overlapping member unfastened.

FIG. 32 is a first side view of the heel float therapeutic boot shown in FIG. 23 with the overlapping member unfastened.

FIG. 33 is a second side view of the heel float therapeutic boot shown in FIG. 23 with the overlapping member unfastened.

FIG. 34 is a perspective view of the heel float therapeutic boot shown in FIG. 23 with an overlapping foot member unfastened.

FIG. 35 is a perspective view of the heel float therapeutic boot shown in FIG. 23 with the overlapping foot member unfastened and a toe tab flexed in.

FIG. 36 is a perspective view of the heel float therapeutic boot shown in FIG. 23 with the overlapping foot member partially unfastened.

FIG. 37 is a first planar view of the heel float therapeutic boot shown in FIG. 23.

FIG. 38 is a second planar view (the other side of the first planar view in FIG. 37) of the heel float therapeutic boot shown in FIG. 23.

FIG. 39 is a front view of an example set of foot support device.

FIG. 40 is a rear view of the set of foot support device of FIG. 39.

FIG. 41 is a front view of the foot support device of FIG. 39.

FIG. 42 is a rear view of the foot support device of FIG. 41.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims

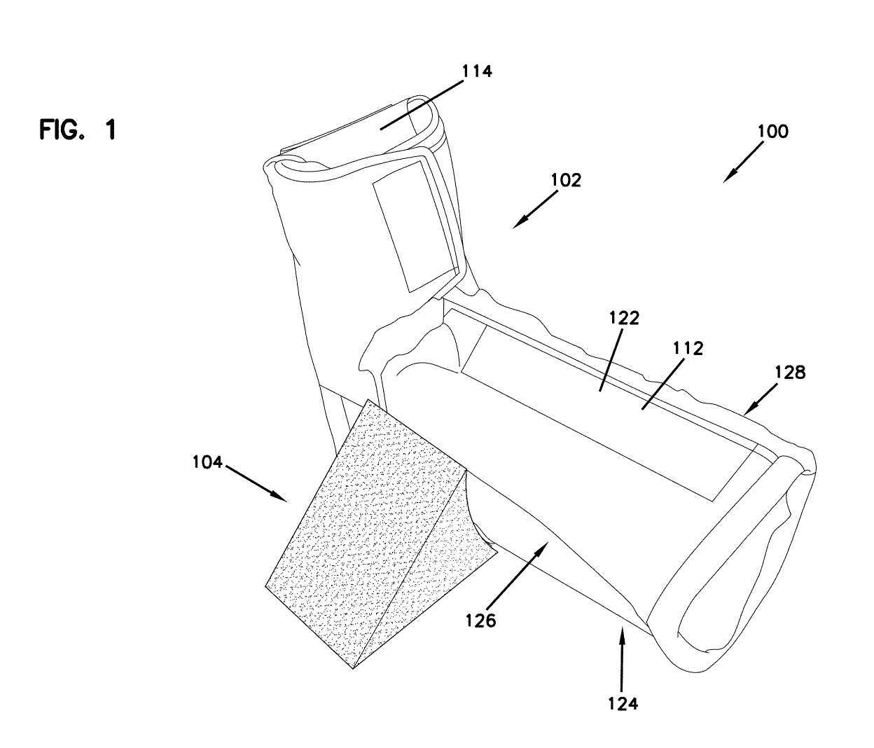

FIG. 1 is a perspective view of an example of a footwear apparatus 100. In this example, the footwear apparatus 100 includes a boot 102 and a support device 104. In some embodiments, the boot 102 includes a lower leg support portion 112 and a foot support portion 114.

The boot 102 can be worn by hospital patients to help support the foot of a patient. In some embodiments, the boot 102 is configured as a heel float therapeutic boot, which is configured to off-load weight from the heel of the patient. The heel float therapeutic boot 102 may help to prevent foot drop by keeping the foot in an upright position thereby preventing the foot from falling over to one side or from pointing toward the foot of the bed while lying down. The heel float therapeutic boot 102 can also be used to elevate the heel so that it does not rub on the bottom of the boot or on the padding. The heel float therapeutic boot 102 supports the foot in a preferred position. Although the boot 102 is primarily described as a heel float therapeutic boot in this document, the boot 102 is not limited to the illustrated configurations and can have other configurations in other embodiments.

The lower leg support portion 112 can be considered as a main body of the boot 102 and typically covers at least a part of the lower leg portion of a patient's leg including at least a portion of the calf and ankle. The lower leg support portion 112 helps protect against pressure ulcers formed on the skin and keeps the legs warm.

In some embodiments, the lower leg support portion 112 has a front wall 122, a back wall 124, and opposite side walls 126, 128. As described herein, the front wall 122 can at least partially surround a front portion of the lower leg. The back wall 124 can at least partially surround a back portion of the lower leg. The side walls 126, 128 can at least partially surround sides of the lower leg.

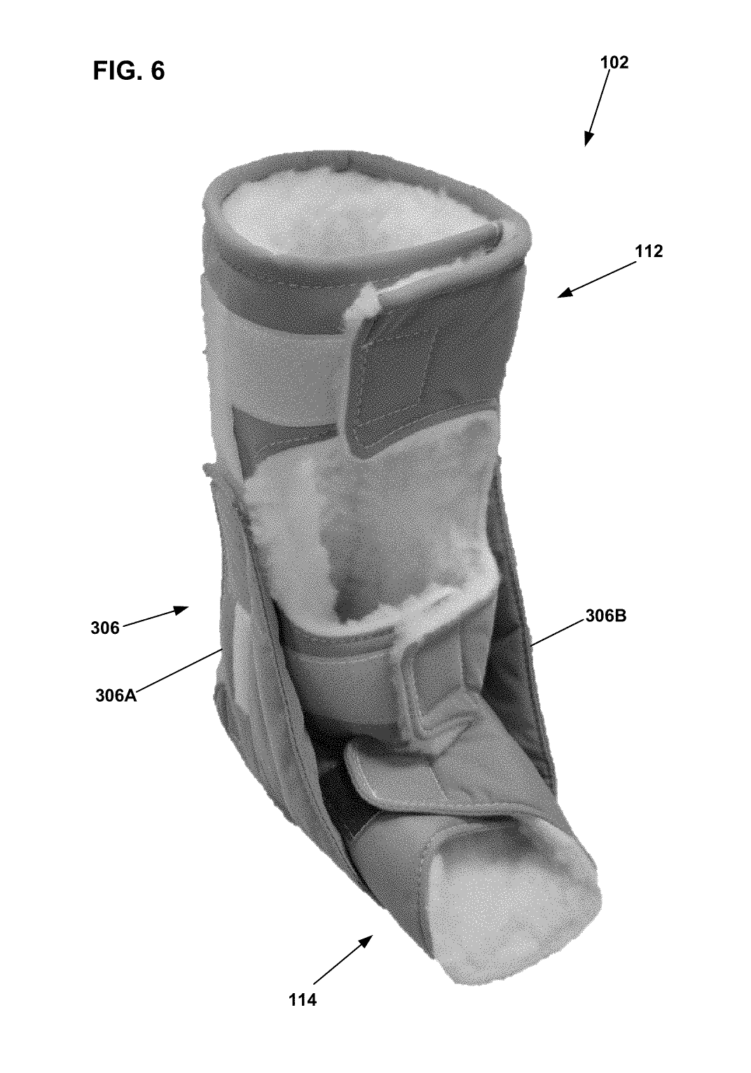

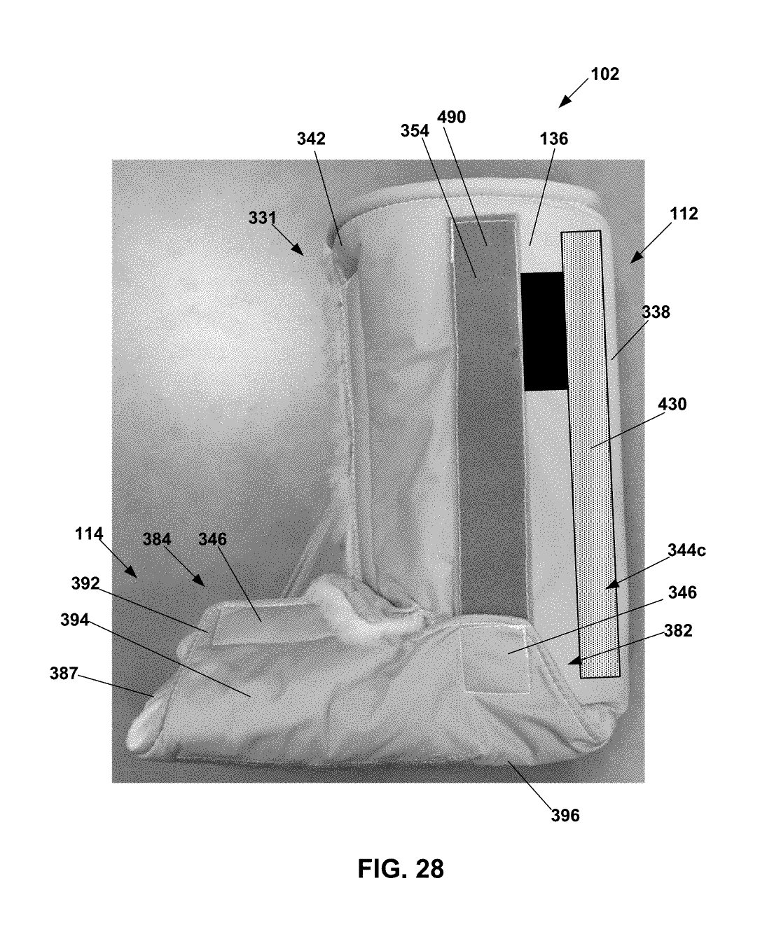

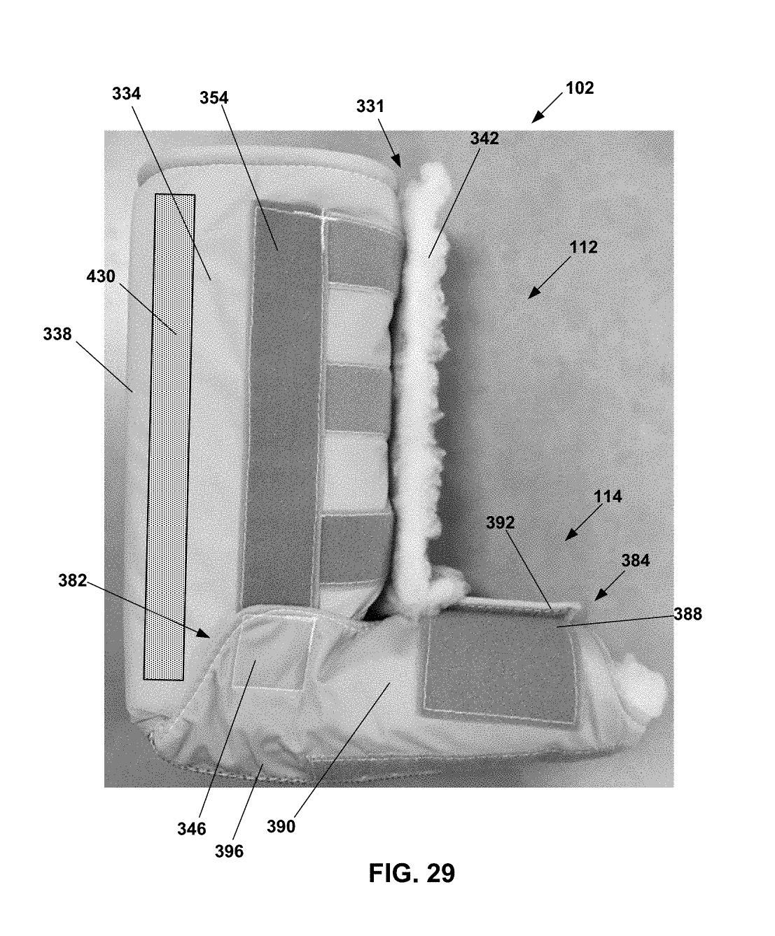

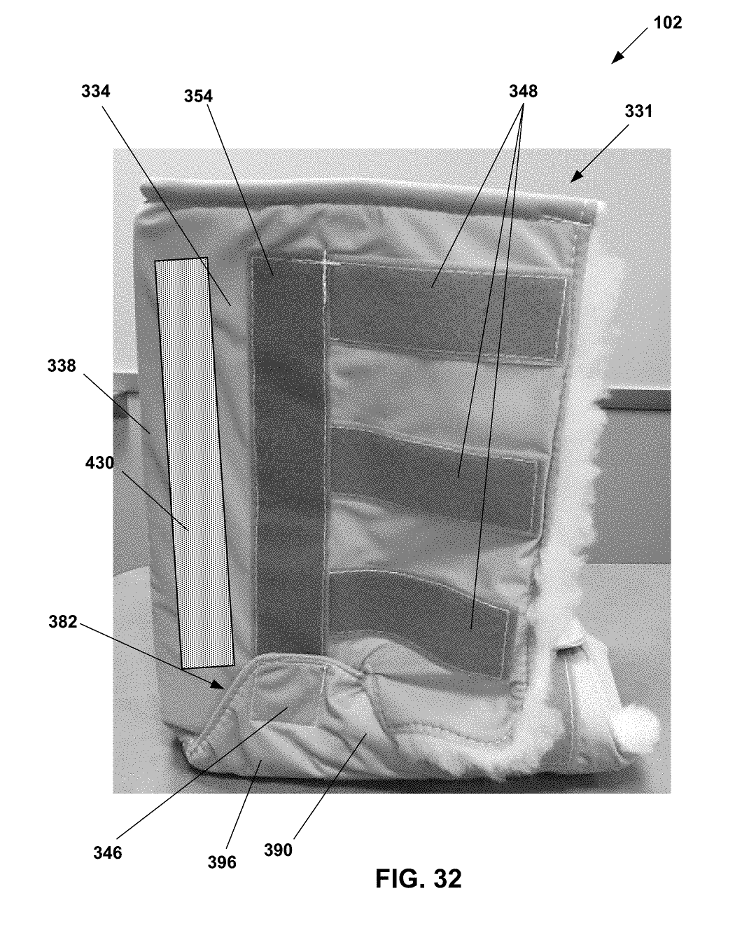

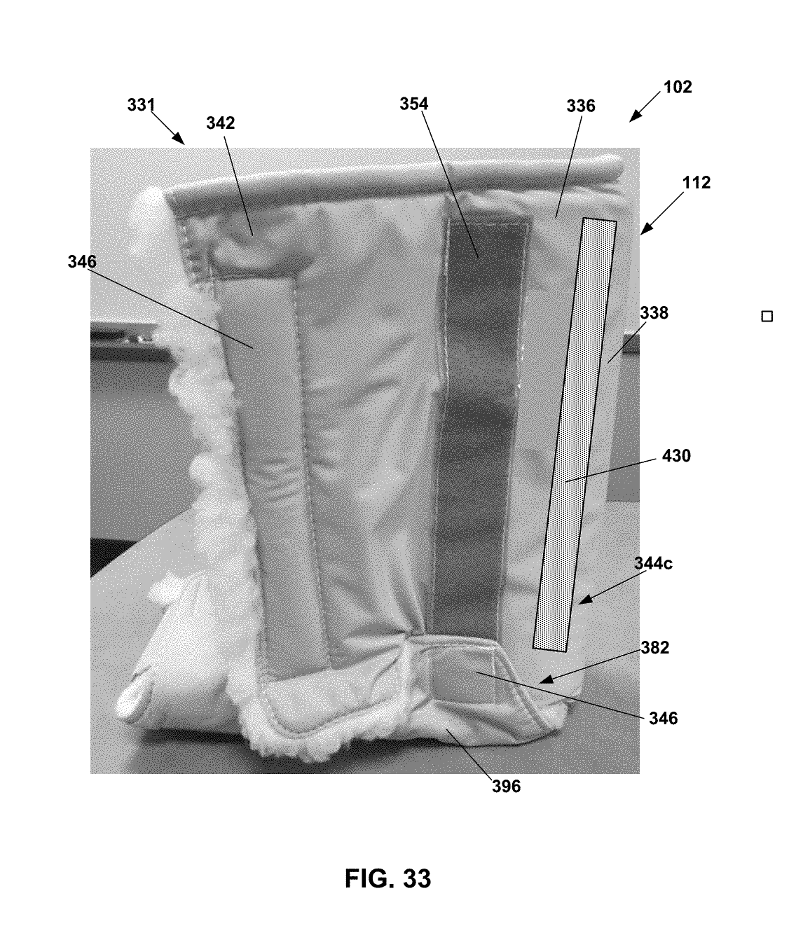

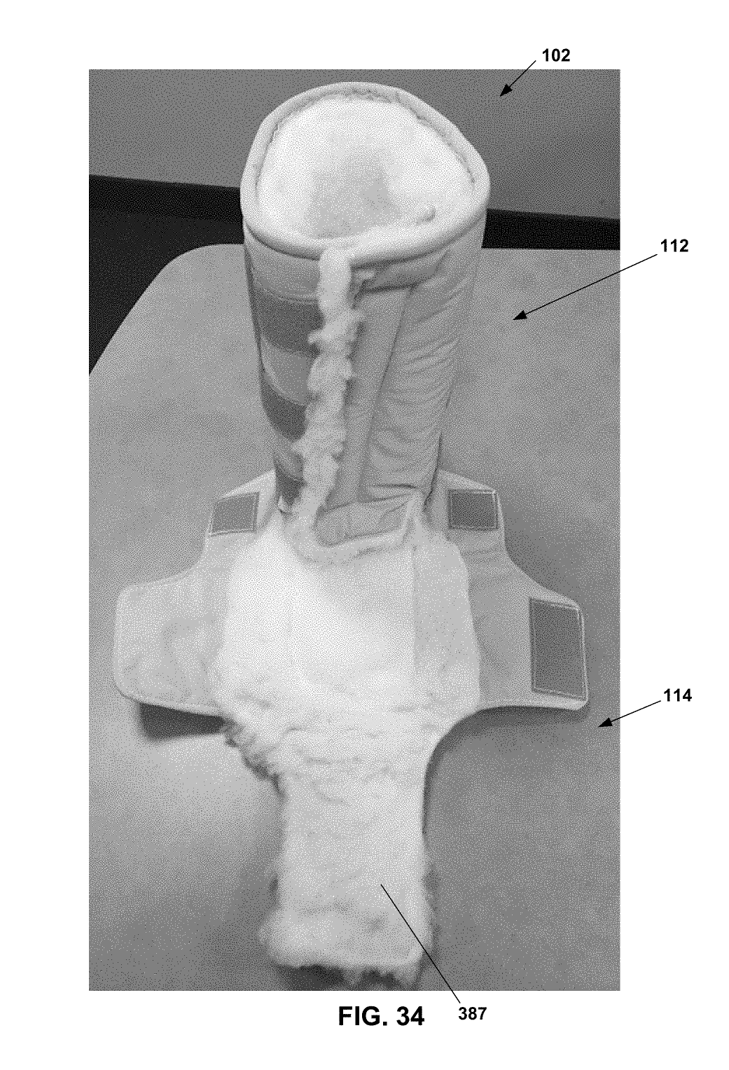

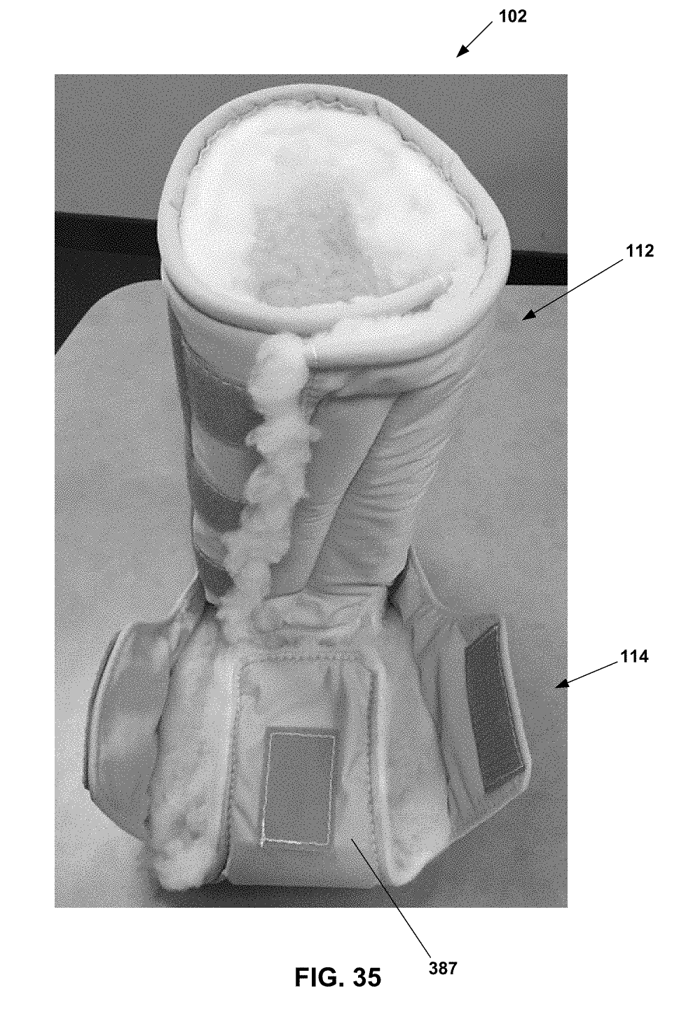

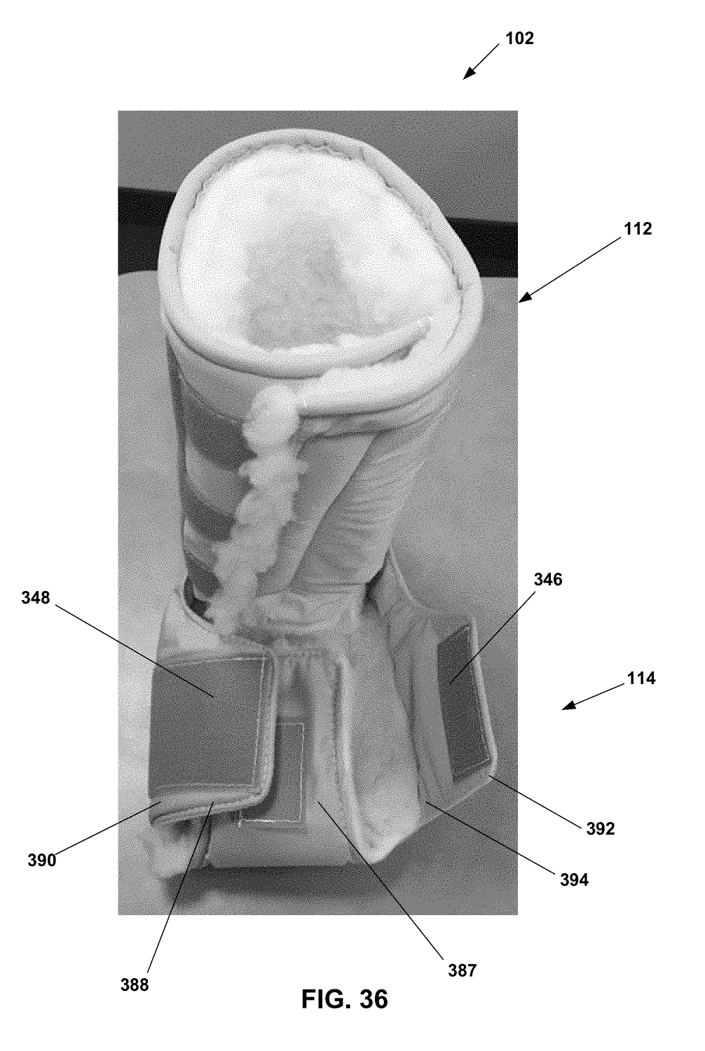

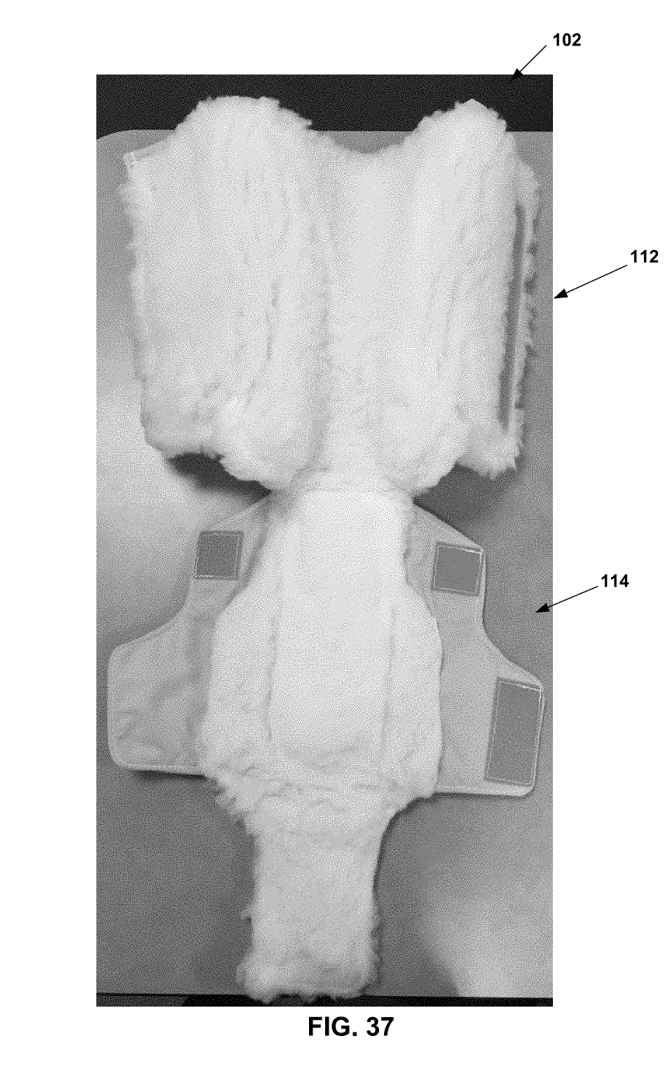

The foot support portion 114 of the boot 102 extends adjacent to the lower leg support portion 112 and covers at least a part of the foot area. The foot support portion 114 helps protect against pressure ulcers formed on the skin and keeps the feet warm. In some embodiments, the foot support portion 114 is connected to the lower leg support portion 112 creating a hinge between the lower leg support portion 112 and the foot support portion 114. Examples of the boot 102 including the lower leg support portion 112 and the foot support portion 114 are described and illustrated in more detail with reference to FIGS. 6-42.

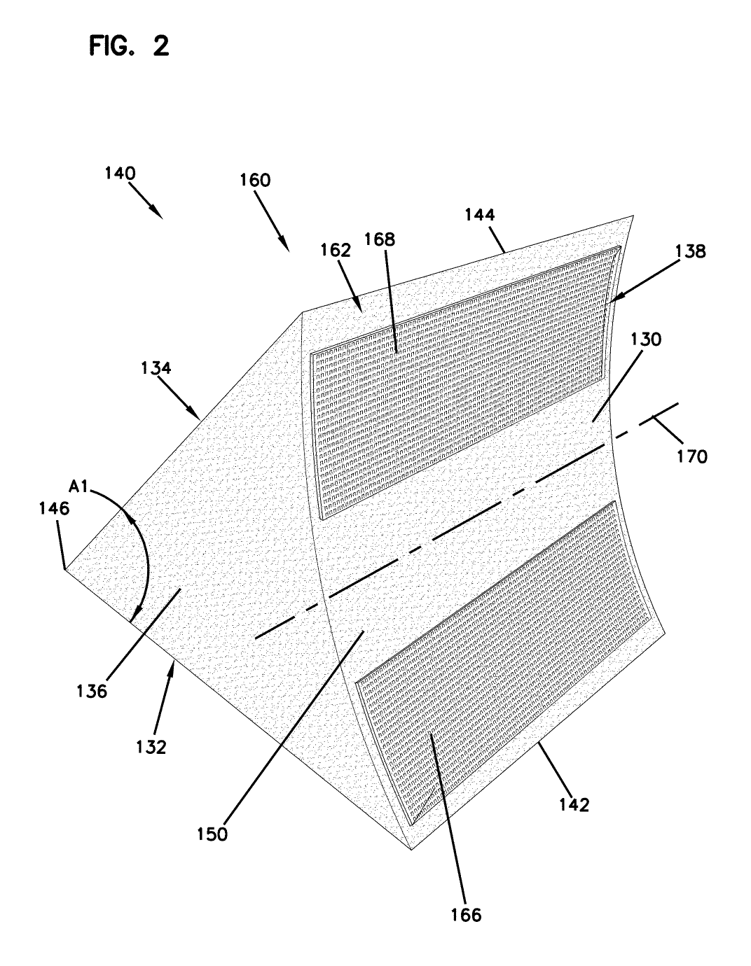

The support device 104 is configured to be arranged with respect to the boot 102 to prevent the boot 102 from tilting or twisting aside over a predetermined position or arrangement. The support device 104 functions to limit such a rotational movement of the boot 102. Further, the support device 104 can set the boot 102 at different positions or arrangements. An example of the support device 104 is described and illustrated in more detail with reference to FIG. 2-5.

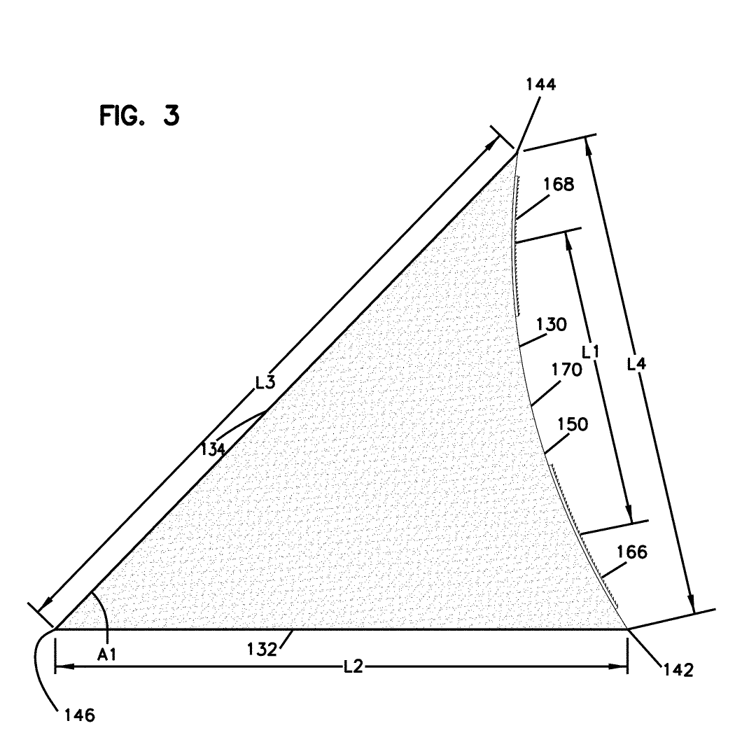

Referring to FIGS. 2 and 3, an example of the support device 104 is further described. In particular, FIG. 2 is a perspective view of an example of the support device 104, and FIG. 3 is a side view of the support device 104.

In some embodiments, the support device 104 is a lateral support device that laterally supports the boot 102. For example, the support device 104 can be used to laterally support the lower leg support portion 112 of the boot 102.

In some embodiments, the support device 104 is generally shaped as a wedge which is defined by two generally triangular sides and three generally quadrilateral faces. As illustrated, the support device 104 has a mounting face 130, a first base face 132, and a second base face 134. The mounting face 130, the first base face 132, and the second base face 134 are generally shaped as quadrilaterals. In the illustrated example, the mounting face 130, the first base face 132, and the second base face 134 have rectangular shapes in general.

The mounting face 130, the first base face 132, and the second base face 134 extend between a first side 136 and a second side 138. The first side 136 and the second side 138 are generally shaped as triangles. In some embodiments, the first side 136 and the second side 138 are arranged in parallel. In other embodiments, the first side 136 and the second side 138 can be arranged at an angle.

The mounting face 130 is abutted to the first base face 132 at a first edge 142, and abutted to the second base face 134 at a second edge 144. The first base face 132 and the second base face 134 are abutted to each other at a third edge 146. The first base face 132 and the second base face 134 are arranged at an angle A1. As illustrated, the angle A1 is an acute angle. As described herein, in some embodiments, the angle A1 ranges between about 20 degrees and about 80 degrees. In other embodiments, the angle A1 ranges between about 30 degrees and about 60 degrees.

Referring to FIG. 3, the first base face 132 extends over a distance L2 between the first edge 142 and the third edge 146. The second base face 134 extends over a distance L3 between the second edge 144 and the third edge 146. The mounting face 130 extends over a distance L4 between the first edge 142 and the second edge 144. By way of example, the distance L2 ranges from about 5.0 inches and about 7.0 inches, the distance L3 ranges from about 5.5 inches and about 8.0 inches, and the distance L4 ranges from about 4.0 inches and about 6.0. In other examples, the distance L2 ranges from about 5.75 inches and about 6.5 inches, the distance L3 ranges from about 6.25 inches and about 7.0 inches, and the distance L4 ranges from about 4.75 inches and about 5.25. Other dimensions are also possible in other embodiments. In some embodiments, the ratios between the distances L2, L3, and L4, such as a ratio of L2 to L3, a ratio of L2 to L4, and a ratio of L3 to L4, can be maintained the same or similar (so that at least one of the angles between at the first, second, and third edges remain the same or similar) while the absolute distances L2, L3, and L4 can change. In other embodiments, the ratios can change.

The mounting face 130 of the support device 104 is configured to be abutted to a side of the boot 102, such as either of the side walls 126, 128 of the lower leg support portion 112. In some embodiments, the mounting face 130 has a shape complementary to a shape of the side of the boot 102. For example, the mounting face 130 has a shape corresponding to the side walls of the lower leg support portion 112.

In some embodiments, the mounting face 130 includes a curved surface 150 for accommodating a curved outline of the lower leg support portion 112 of the boot 102. When the lower leg support portion 112 wraps a patient's lower leg, the lower leg support portion 112 can generally form a rounded shape having a convex exterior. To effectively support the rounded shape of the lower leg support portion 112, the curved surface 150 of the mounting face 130 has a concave shape complementary to the convex exterior of the lower leg support portion 112.

In some embodiments, the support device 104 is configured to be removably arranged to a side of the boot 102, as illustrated in FIG. 1. For example, the support device 104 is detachably mounted to either of the side walls 136, 138 of the lower leg support portion 112.

Referring to FIG. 2, in some embodiments, the footwear apparatus 100 includes a fastening device 160 for detachably mounting the mounting face 130 of the support device 104 to the boot 102. For example, the fastening device 106 is configured to mount the mounting face 130 of the support device 104 to either of the side walls 136, 138 of the lower leg support portion 112.

In some embodiments, the fastening device 160 can include a support device fastener 162 and a boot fastener 164. The support device fastener 162 is provided to the support device 104 and the boot fastener 164 is provided to the boot 102. The support device fastener 162 can cooperate with the boot fastener 164 to attach the support device 104 to the boot 102.

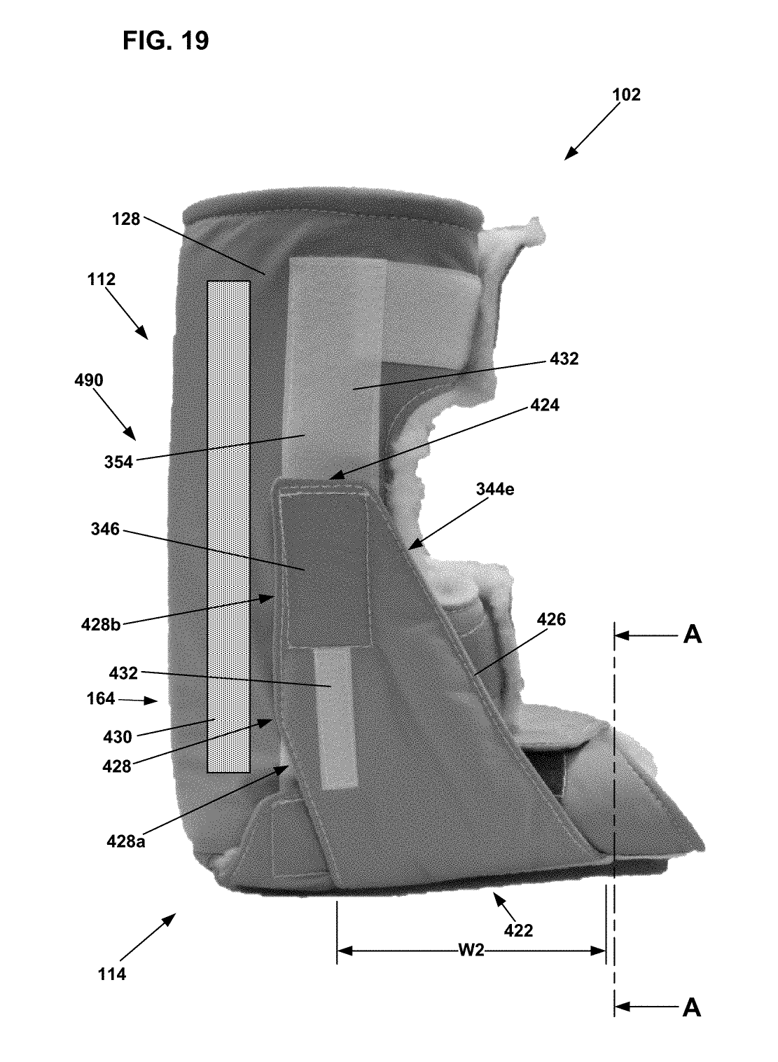

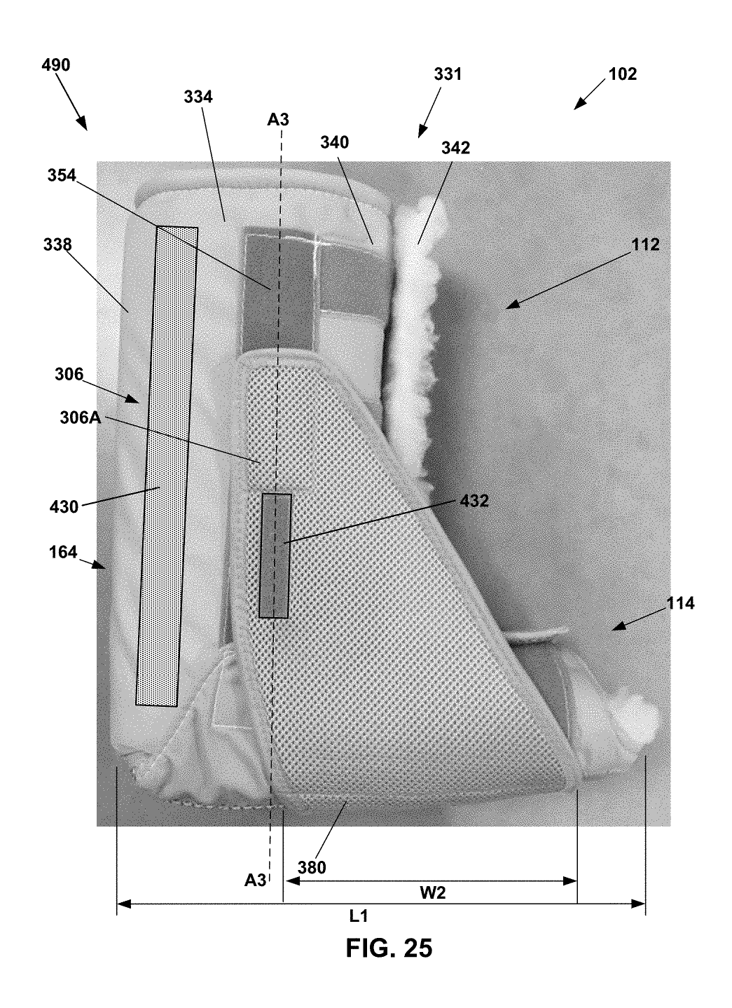

In one example, the support device fastener 162, which is provided to the support device 104, includes one or more support device fastening patches. In the illustrated example, a pair of support device fastening patches, such as a first support device fastening patch 166 and a second support device fastening patch 168, is provided on the mounting face 130 of the support device 104. As described below, the boot fastener 164 can include boot fastening patches corresponding to the support device fastening patches. As illustrated in FIGS. 19 and 25, the boot 102 includes a first boot fastening patch 430 and a second boot fastening patch 432, which are arranged to correspond to the first support device fastening patch 166 and the second support device fastening patch 168. As described below, the first boot fastening patch 430 and the second boot fastening patch 432 are arranged on the side walls 126, 128 of the lower leg support portion 112, so that the support device 104 laterally supports the lower leg support portion 112 of the boot 102.

The fastening device 160 can be of various types. In one example, the fastening device 160 includes hook-and-loop fasteners, such as Velcro®, available from Velcro Companies (UK). For example, the support device fastening patches 166, 168 and the boot fastening patches 430, 432 can include hook patches and loop patches. By way of example, the support device fastening patches 166, 168 are formed as hook patches and the boot fastening patches 430, 432 are made as loop patches. The other configurations are also possible.

With still reference to FIGS. 2 and 3, the first support device fastening patch 166 and the second support device fastening patch 168 are arranged such that a center line 170 positioned at the middle between the first edge 142 and the second edge 144 is located between the first support device fastening patch 166 and the second support device fastening patch 168. In some embodiments, the first support device fastening patch 166 and the second support device fastening patch 168 are arranged adjacent to the first edge 142 and the second edge 144, respectively. In other embodiments, the first support device fastening patch 166 is spaced apart from the second support device fastening patch 168 at a distance L1, which can be measured between the center of the first support device fastening patch and the center of the second support device fastening patch. In some embodiments, the distance L1 can range from about 1.5 inches to about 4.5 inches. In other embodiments, the distance L1 can range from about 2.5 inches to about 3.5 inches. Other measurements for the distance L1 are also possible.

In other embodiments, the support device fastener 162 includes a single support device fastening patch and the boot fastener 164 includes one or more boot fastening patches 430, 432, so that the single fastening patch of the support device fastener 162 is detachably attached to the one or more boot fastening patches 430, 432 of the boot fastener 164. In yet other embodiments, the support device fastener 162 includes a plurality of fastening patches while the boot fastener 164 includes a single boot fastening patch on which the at least one of the plurality of fastening patches of the support device fastener 162 can be removably attached. In yet other embodiments, the support device fastener 162 includes more than three support device fastening patches and the boot fastener 164 includes more than three support device fastening patches corresponding to the support device fastening patches of the support device fastener 162. In yet other embodiments, the support device fastener 162 and the boot fastener 164 have different configurations.

In other examples, the fastening device 160 is made with other types, such as buttons, snaps, zip fasteners, adhesive, hooks, clips, clasps, bolts, straps, or combinations of these or other fastening mechanisms.

The support device 104 can be made of various materials. In some embodiments, the support device 104 is made of polyurethane open cell form. Other various materials can be used to make the support device 104 in other embodiments.

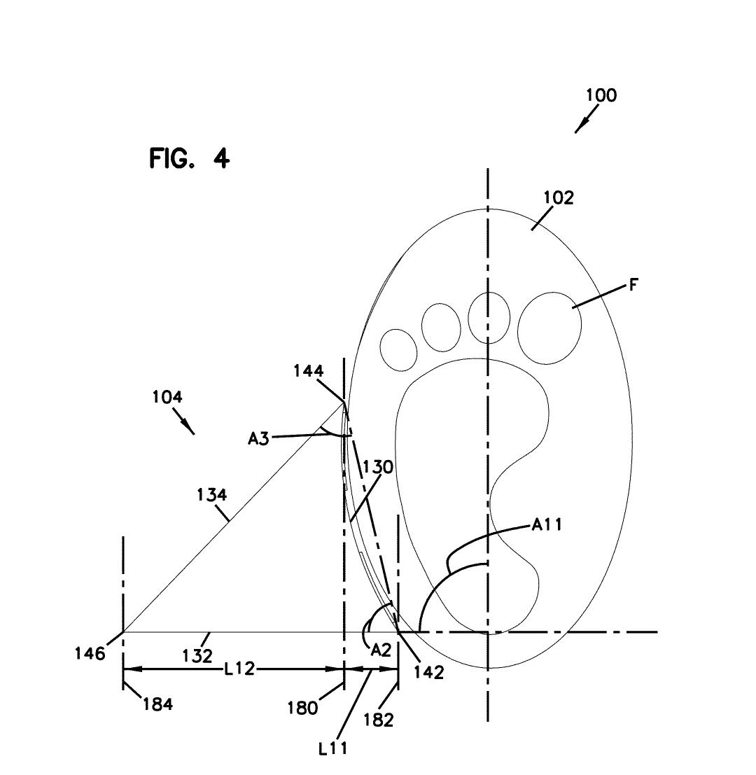

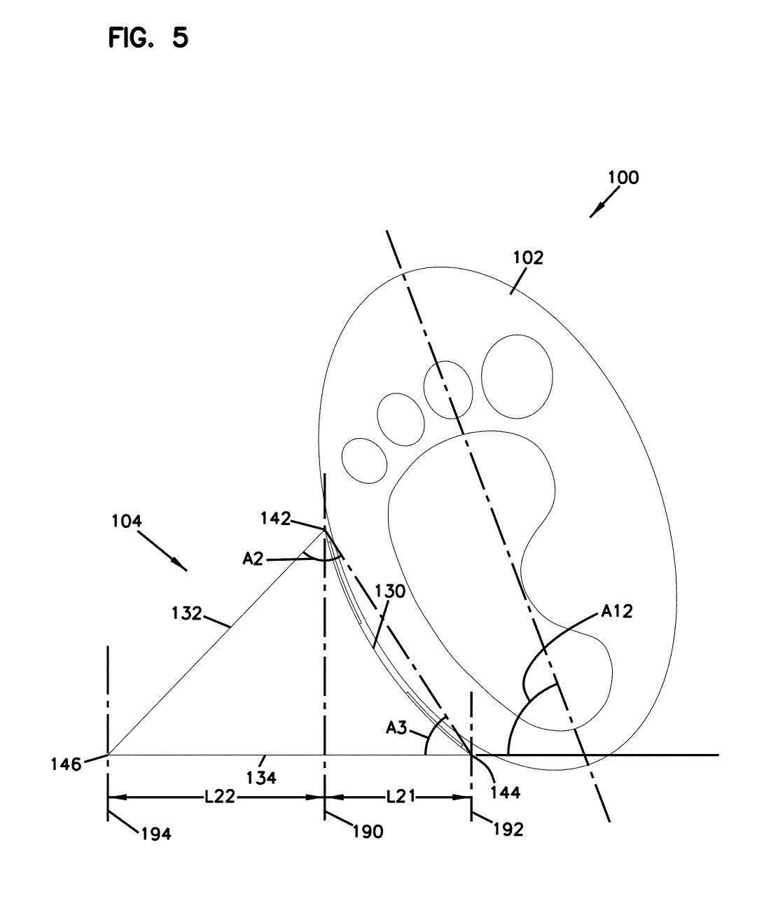

Referring to FIGS. 4 and 5, it is illustrated that the support device 104 can be used to support the boot 102 at two different positions. In particular, FIG. 4 illustrates that the support device 104 is arranged in a first position (or a first orientation) to support the boot 102 at a first angle, and FIG. 5 illustrates that the support device 104 is arranged in a second position (or a second orientation) to support the boot 102 at a second angle.

The support device 104 can support the boot 102 in different positions by arranging the support device 104 in different arrangements relative to the boot 102. In some embodiments, the support device 104 can be arranged such that either of the first base face 132 or the second base face 134 is used as a bottom of the support device 104. In one example, as illustrated in FIG. 4, the support device 104 is arranged on a ground surface (e.g., a patient bed surface, a floor, and other surfaces on which a patient lies) such that the first base face 132 is positioned as a bottom of the support device 104. In another example, as illustrated in FIG. 5, the support device 104 is arranged on the ground surface such that the second base face 134 is positioned as the bottom of the support device 104.

In some embodiments, the length L2 of the first base face 132 (i.e., the distance L2 between the first edge 142 and the third edge 146) is different from the length L3 of the second base face 134 (i.e., the distance L3 between the second edge 144 and the third edge 146). In one example, the length L2 of the first base face 132 is shorter than the length L3 of the second base face 134. In other examples, the length L2 of the first base face 132 and the length L3 of the second base face 134 define a predetermined ratio therebetween. In some embodiments, a ratio of the length L2 of the first base face 132 to the length L3 of the second base face 134 ranges between about 1:1.05 and about 1:1.30. In other embodiments, the ratio of the length L2 of the first base face 132 to the length L3 of the second base face 134 ranges between about 1:1.10 and about 1:1.20. In yet other embodiments, other ratios are also possible.

As described further below, the difference between the length L2 and the length L3 provides different angles at which the boot 102 is supported by the support device 104. Because the length L2 and the length L3 are different, an angle A2 defined by the first base face 132 and the mounting face 130 at the first edge 142 is different from an angle A3 defined by the second base face 134 and the mounting face 130 at the second edge 144. Such different angles A2 and A3 provide different angles of the mounting face 130 relative to the boot 102, depending on which of the first base face 132 and the second base face 134 is used as the bottom of the support device 104. In some embodiments, the angle A2 ranges between about 20 degrees and about 80 degrees. In other embodiments, the angle A2 ranges between about 40 degrees and about 60 degrees. In some embodiments, the angle A3 ranges between about 20 degrees and about 80 degrees. In other embodiments, the angle A2 ranges between about 40 degrees and about 60 degrees.

As illustrated in FIG. 4, the support device 104 can be arranged in a first position. In the first position, the first base face 132 is arranged as a bottom of the support device 104 and laid on a ground surface, such as a patient bed surface, a floor, and other surfaces on which a patient lies. In the first position, the mounting face 130 of the support device 104 is positioned to support the lower leg support portion 112 of the boot 102 in a first arrangement and prevent the boot 102 from tilting or twisting aside over the first arrangement. When the support device 104 is in the first position, the boot 102 (e.g., the foot support portion 114 thereof) is oriented at a first boot angle A11 relative to the ground surface. In some embodiments, the first boot angle A11 can range between about 60 degrees and about 120 degrees. In other embodiments, the first boot angle A11 can range between about 70 degrees and about 100 degrees. In yet other embodiments, the first boot angle A11 can be about the right angle so that the foot F wrapped by the foot support portion 114 is oriented substantially vertically with respect to the ground surface.

In the first position, the support device 104 is configured such that a first imaginary plane 180 orthogonal to the bottom (i.e., the first base face 132 in the first position) and including the second edge 144 is positioned between a second imaginary plane 182 orthogonal to the bottom and including the first edge 142 of the mounting face 130 and a third imaginary plane 184 orthogonal to the bottom and including the third edge 146.

The first imaginary plane 180 and the second imaginary plane 182 are spaced apart at a first distance L11, and the first imaginary plane 180 and the third imaginary plane 184 are spaced apart at a second distance L12. In some embodiments, a ratio of the first distance L11 to the second distance L12 ranges between about 1:2 and about 1:5. In other embodiments, a ratio of the first distance L11 to the second distance L12 ranges between about 1:3 and about 1:4. In yet other embodiments, other ratios are also possible.

As illustrated in FIG. 5, the support device 104 can be alternatively arranged in a second position. In the second position, the second base face 134 is positioned as the bottom of the support device 104 and laid on the ground surface. In the second position, the mounting face 130 of the support device 104 is positioned to support the lower leg support portion 112 of the boot 102 in a second arrangement and prevent the boot 102 from tilting or twisting aside over the second arrangement. When the support device 104 is in the second position, the boot 102 (e.g., the foot support portion 114 thereof) is oriented at a second boot angle A12 relative to the ground surface. In some embodiments, the second boot angle A12 can range between about 30 degrees and about 90 degrees. In other embodiments, the first boot angle A11 can range between about 50 degrees and about 70 degrees.

In the illustrated example, when the support device 104 is in the second position, the lower leg support portion 112 of the boot 102 remains more tilted in the second arrangement (FIG. 5) than in the first arrangement (FIG. 4). In some embodiments, a patient's leg or foot can naturally tilt outwardly to some extent when the patient lies on a patient bed. The second position of the support device 104 is designed to accommodate such a natural tilting angle of the boot 102 while providing sufficient lateral support to the boot 102 at that angle.

In the second position, a fourth imaginary plane 190 orthogonal to the bottom (i.e., the second base face 134 in the second position) and including the first edge 142 is positioned between a fifth imaginary plane 192 orthogonal to the bottom and including the second edge 144 and a sixth imaginary plane 194 orthogonal to the bottom and including the third edge 146.

The forth imaginary plane 190 and the fifth imaginary plane 192 are spaced apart at a third distance L21, and the fourth imaginary plane 190 and the sixth imaginary plane 194 are spaced apart at a fourth distance L22. In some embodiments, a ratio of the third distance L21 to the fourth distance L22 ranges between about 1:1.3 and about 1:2.5. In other embodiments, the ratio of the third distance L21 to the fourth distance L22 ranges between about 1:1.5 and about 1:2. In yet other embodiments, other ratios are also possible.

Referring to FIGS. 6-22, an example of the boot 102 is further described.

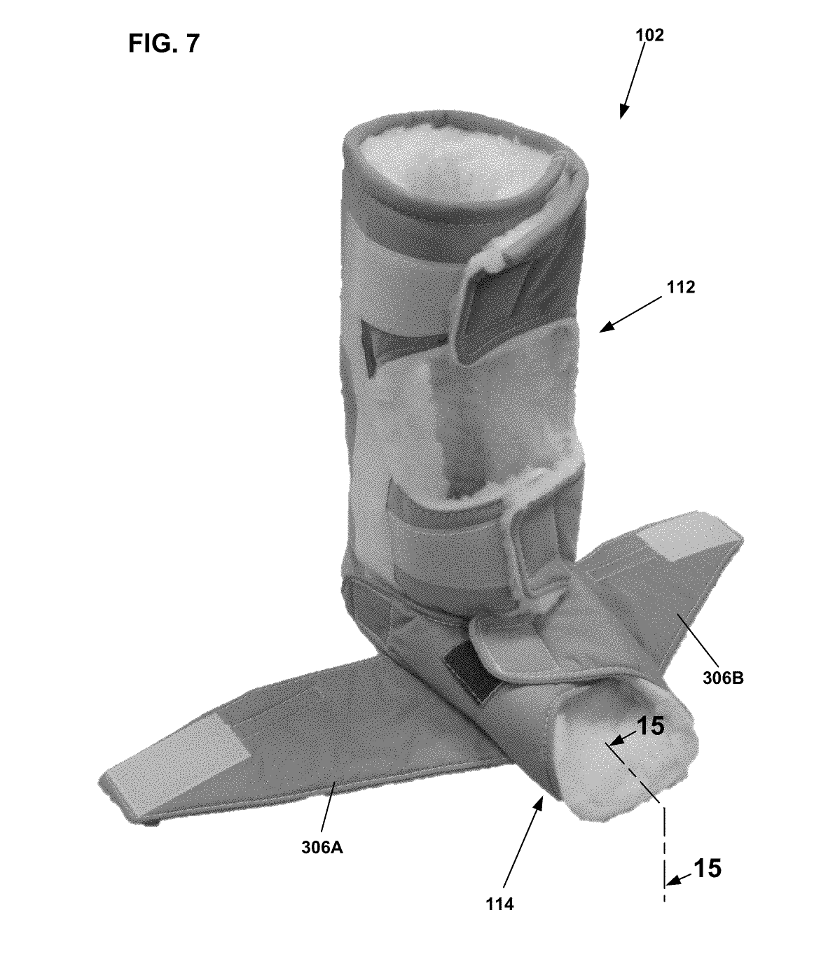

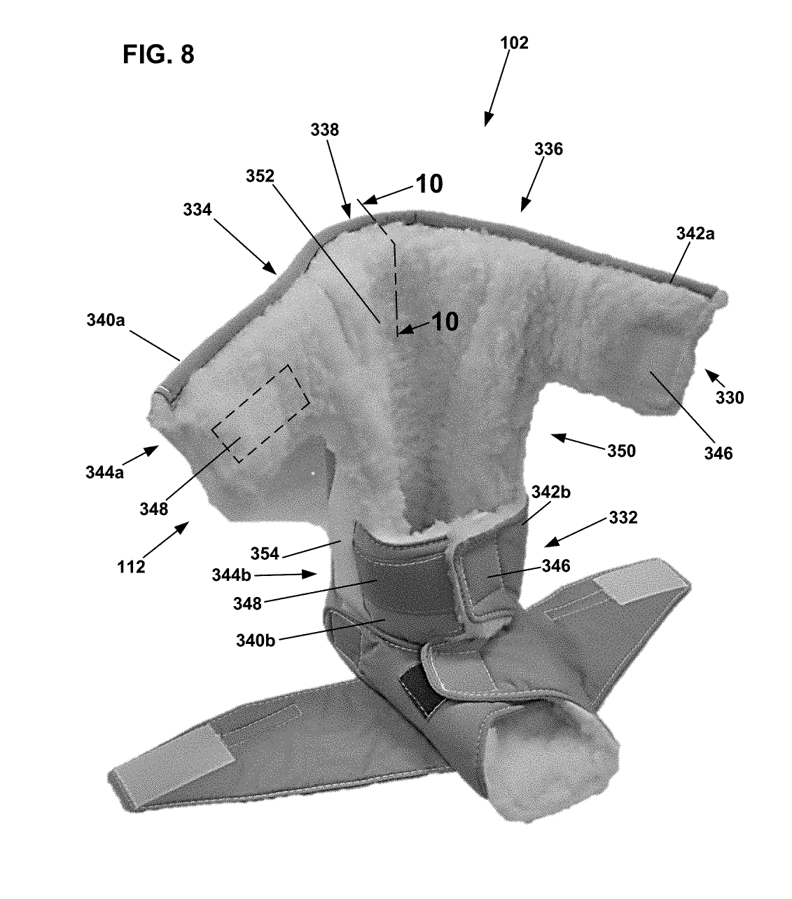

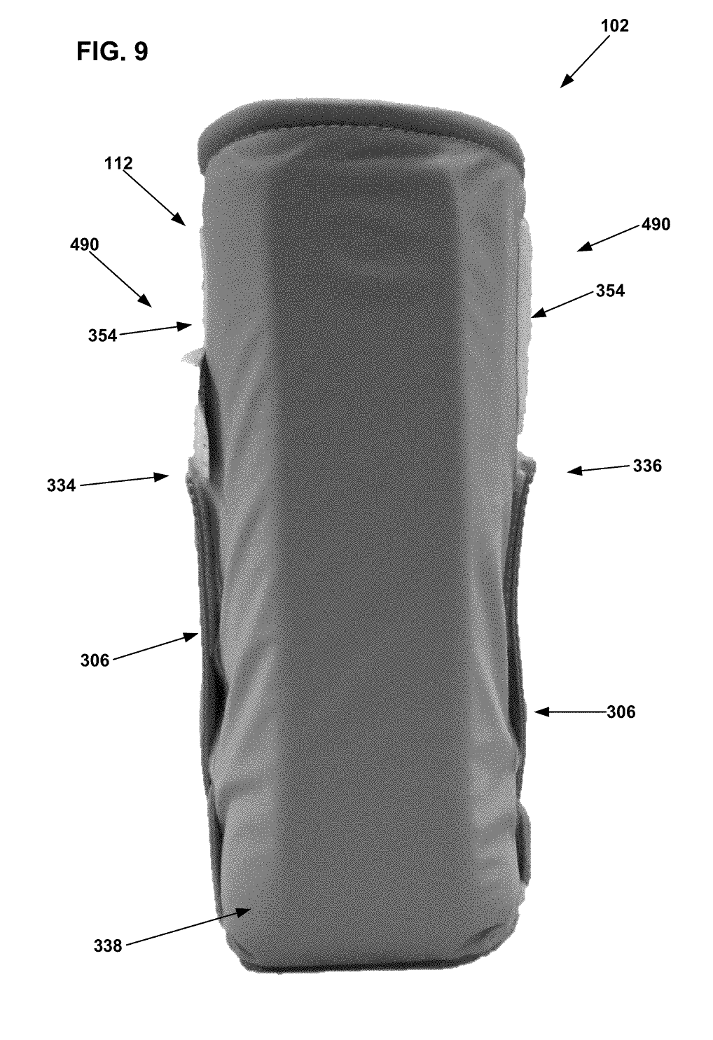

FIGS. 6-7 are perspective views of an example of the boot 102. In some embodiments, the boot 102 is configured as a heel float therapeutic boot, as described herein. In this document, the boot 102 is also referred to as the heel float therapeutic boot or heel float therapeutic footwear apparatus. In this example, in addition to the lower leg support portion 112 and the foot support portion 114, the boot 102 includes a wing support structure 306. In FIG. 8, the wing support structure 306 is configured as being attached to the boot 102. In FIG. 9, the wing support structure 306 is shown as being open and unfastened to the boot 102.

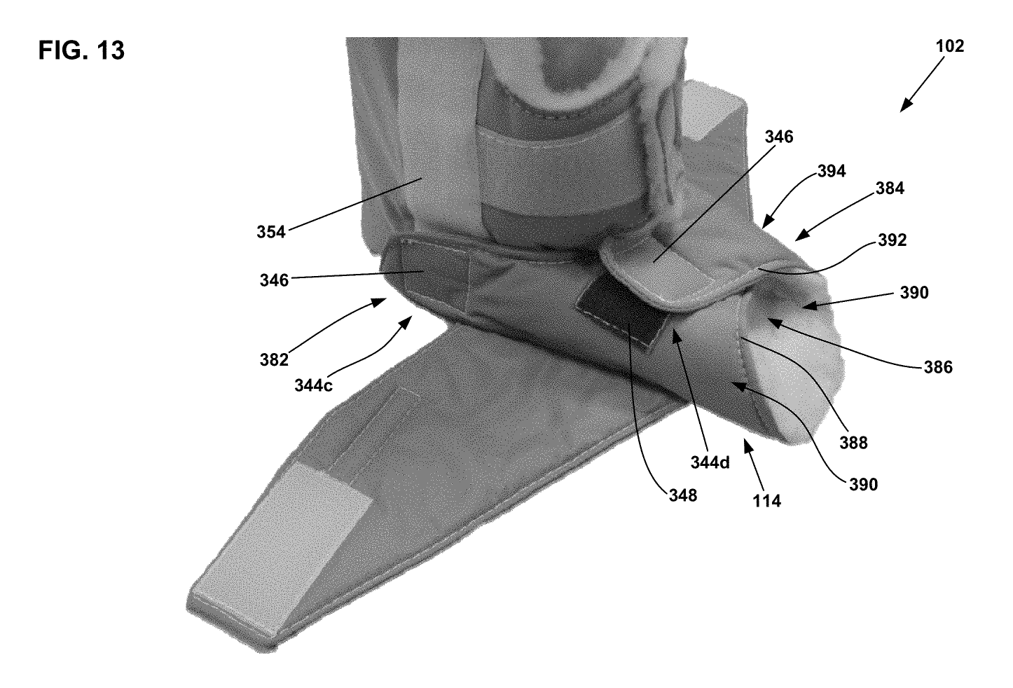

As described herein, the lower leg support portion 112 is configured to at least partially wrap a lower leg, and the foot support portion 114 extends from the lower leg support portion and covers at least a part of the foot area. The lower leg support portion 112 is illustrated and described in more detail with reference to FIGS. 8-12, and the foot support portion 114 is illustrated and described in more detail with reference to FIGS. 13-17.

The wing support structure 306 of the heel float therapeutic boot 102 supports the foot in a preferred position. The wing support structure 306 is attached to the heel float therapeutic boot 102 and is configured to be adjustable to obtain the preferred position. The wing support structure 306 is illustrated and described in more detail with reference to FIGS. 19-21.

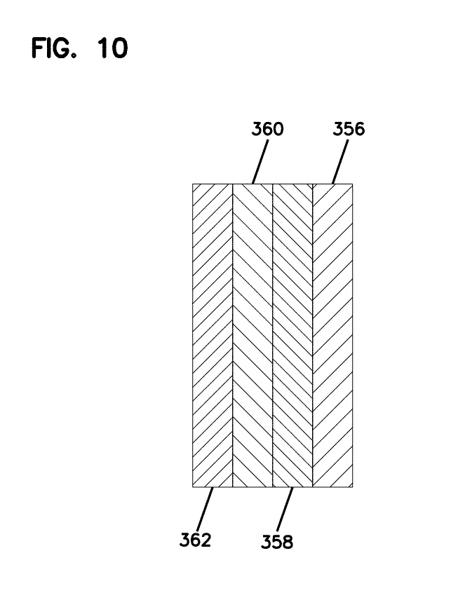

Referring to FIGS. 8-10, various features of the lower leg support portion 112 are illustrated. In particular, FIGS. 8-9 are perspective front and back views of the lower leg support portion 112. In this example, the lower leg support portion 112 includes an upper overlapping member 330, a lower overlapping member 332, a left side wall 334 (e.g., the side wall 126 in FIG. 1), a right side wall 336 (e.g., the side wall 128 in FIG. 1), and a back side wall 338 (e.g., the back wall 124 in FIG. 1). In some embodiments, the lower leg support portion 112 is formed of one piece connecting all of the side walls with both of the overlapping members. In other embodiments, the lower leg support portion 112 is formed of a plurality of pieces which are connected to one another in various manners. The lower leg support portion 112 includes multiple layers that are illustrated and described in detail with reference to FIG. 10.

The upper overlapping member 330 includes a left side panel 340a extending from the left side wall 334 of the lower leg support portion 112 and a right side panel 342a extending from the right side wall 336 of the lower leg support portion 112. The upper overlapping member 330 has a fastening mechanism 344a to connect the left side panel 340a together with the right side panel 342a. In some embodiments, the fastening mechanism 344a includes a hook patch 346 and a loop patch 348. In other embodiments, other fastening mechanisms are used, such as, buttons, adhesive, hooks, clips, clasps, bolts, straps, or combinations of these or other fastening mechanisms. The loop patch 348 is attached to the exterior surface of the left side panel 340a of the upper overlapping member 330 and the hook patch 346 is attached to the right side panel 342a of the upper overlapping member 330. In this example, the hook patch 346 and the loop patch 348 are sewn onto respective panels of the upper overlapping member 330 to be fastened together as the two overlap. In other embodiments, the arrangement and configuration of the hook patch 346 and the loop patch 348 can vary.

The lower overlapping member 332 includes a left side panel 340b extending from the left side wall 334 of the lower leg support portion 112 and a right side panel 342b extending from the right side wall 336 of the lower leg support portion 112. The lower overlapping member 332 has a fastening mechanism 344b to connect the left side panel 340b together with the right side panel 342b. The lower overlapping member 332 may also be provided with a hook patch 346 and a loop patch 348 as the fastening mechanism 344b similar to those described for the upper overlapping member 330. Accordingly, many of the concepts and features for the lower overlapping member 332 are similar to the upper overlapping member 330 previously described herein. In some embodiments, the lower overlapping member 332 is color coded such that the hook patch 346 and loop patch 348 colors are different from other fastening mechanisms, i.e. the upper overlapping member 330. This feature helps to identify for the patient where each overlapping member should be attached if the heel float therapeutic boot 102 becomes crumpled or twisted.

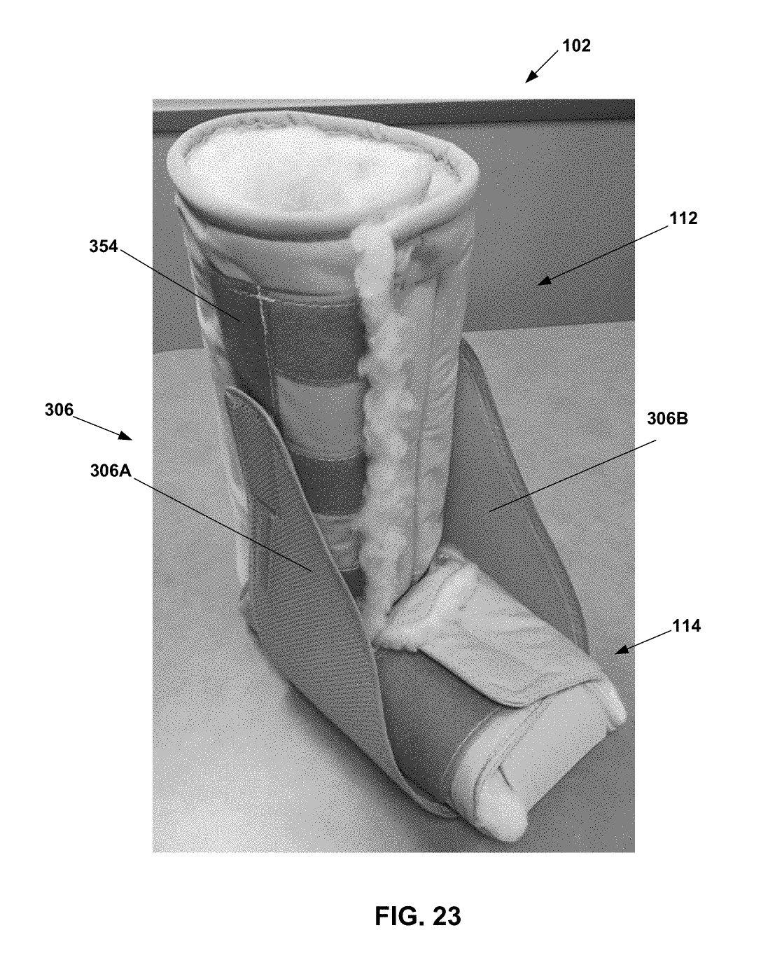

In this example, the upper and lower overlapping members 330, 332 of the lower leg support portion 112 are arranged and configured to wrap and fasten about an anterior portion of the lower leg. In other examples, the lower leg support portion 112 may extend further up the leg. In some embodiments, the upper overlapping member 330 and the lower overlapping member 332 are arranged and configured to define an opening 350 in the front of the lower leg support portion 112 of the heel float therapeutic boot 102. This opening may help provide a cooling feature for the patient when warming is not so critical. It is to be understood that other configurations may be used, such as, but not limited to, a closed configuration having no opening, a slide on boot, or a slip on boot, and combinations of these and other configurations. For example, as illustrated in FIG. 23, the lower leg support portion 112 of the heel float therapeutic boot 102 has a single overlapping member that does not define an opening (such as the opening 350) when the overlapping member is fastened around the lower leg.

Referring to FIG. 9, the left side wall 334 of the lower leg support portion 112 and the right side wall 336 of the lower leg support portion 112 are integrally formed with the back side wall 338 of the lower leg support portion 112 defining a channel 352 (shown in FIG. 3) for receiving a lower leg of a patient. In this example, the left side wall 334 of the lower leg support portion 112 and the right side wall 336 of the lower leg support portion 112 have a leg support attachment element 490. In some embodiments, the leg support attachment element 490 can include a loop material 354 vertically positioned along the left and right side of the lower leg support portion 112. In this example, the wing support structure 306 is configured to fasten along the loop material 354 positioned on the left and right side of the lower leg support portion 112.

FIG. 10 is a component view of a portion of the heel float therapeutic boot 102 depicting layers of the lower leg support portion 112. In this example, the lower leg support portion 112 includes a fleece material 356, a lining 358, a heel float cradle 360 and exterior fabric 362. The construction and materials used in the heel float therapeutic boot 102 provides the proper support and protection needed for patients.

The fleece material 356 includes the entire lining 358 of the heel float therapeutic boot 102 and is positioned to surround the lower leg of the patient. In this example, the fleece material 356 covers the entire interior of the heel float therapeutic boot 102 to provide for a soft, smooth/comfortable surface to contact the skin without any abrading seams that can cause skin ulcerations. In some embodiments, the fleece material 356 is laminated to the lining 358 of the heel float therapeutic boot 102. In other embodiments, the fleece material 356 can be placed on the lining 358 by other attachment means, such as, adhesive, sewing, or combinations of these or other attachment means.

The lining 358 helps wick moisture away and ventilate the lower leg and foot. Moisture typically develops at regions of sustained contact between the skin and the fleece material 356. In some embodiments, seams can be located between sections of the fleece material 356 and/or lining 358 to minimize contact with the skin. These seams can be sewn or adhesively fixed in the heel float therapeutic boot 102.

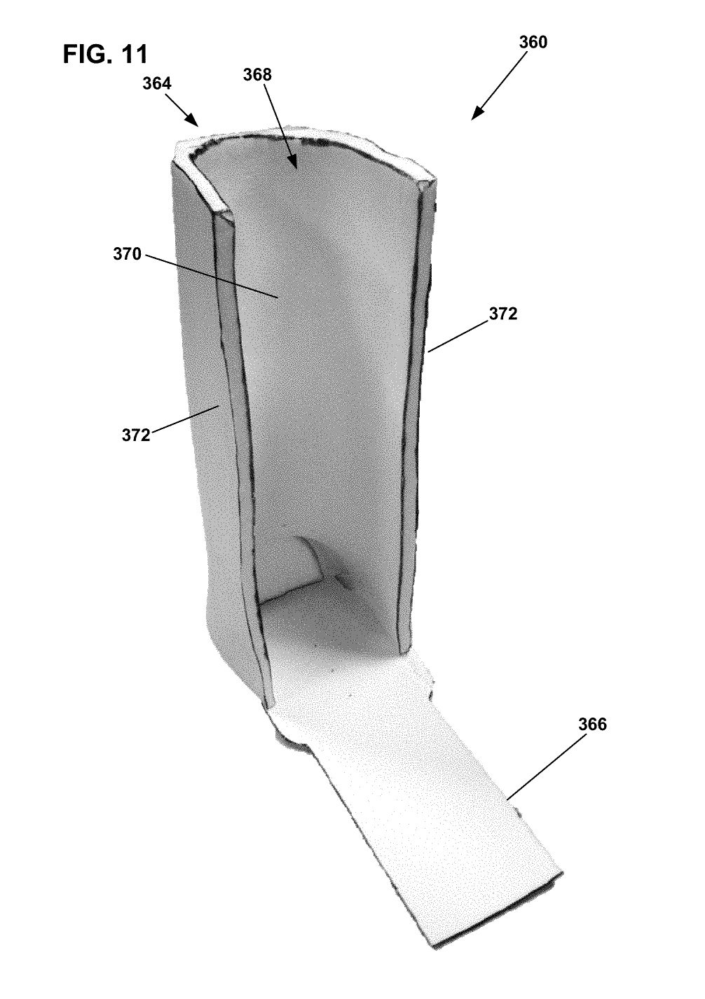

The heel float cradle 360 is typically a relatively dense elastic material that flexes or compresses slightly to provide a resilient interface with the lower leg, for example, foam. In this example, the heel float cradle 360 is positioned between the exterior fabric 362 of the heel float therapeutic boot 102 and the lining 358 of the heel float therapeutic boot 102. In some embodiments, the heel float cradle 360 extends along the length of the lower leg support portion 112 and the foot support portion 114. The heel float cradle 360 is illustrated and described in more detail with reference to FIG. 11.

The exterior fabric 362 is constructed of an air permeable material, for example, a durable velour cloth. In some embodiments, other materials such as a heavy weight cotton fabric, CORDURA® or other fabric or fabric combinations might also be used. The exterior fabric 362 covers the entirety of the heel float therapeutic boot 102.

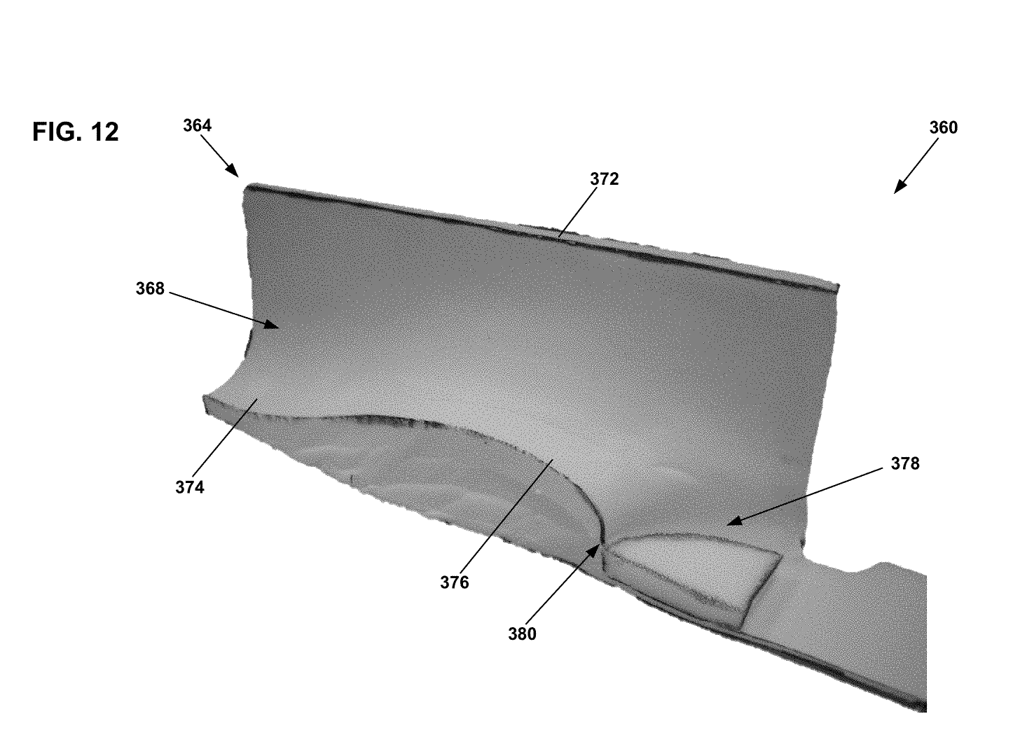

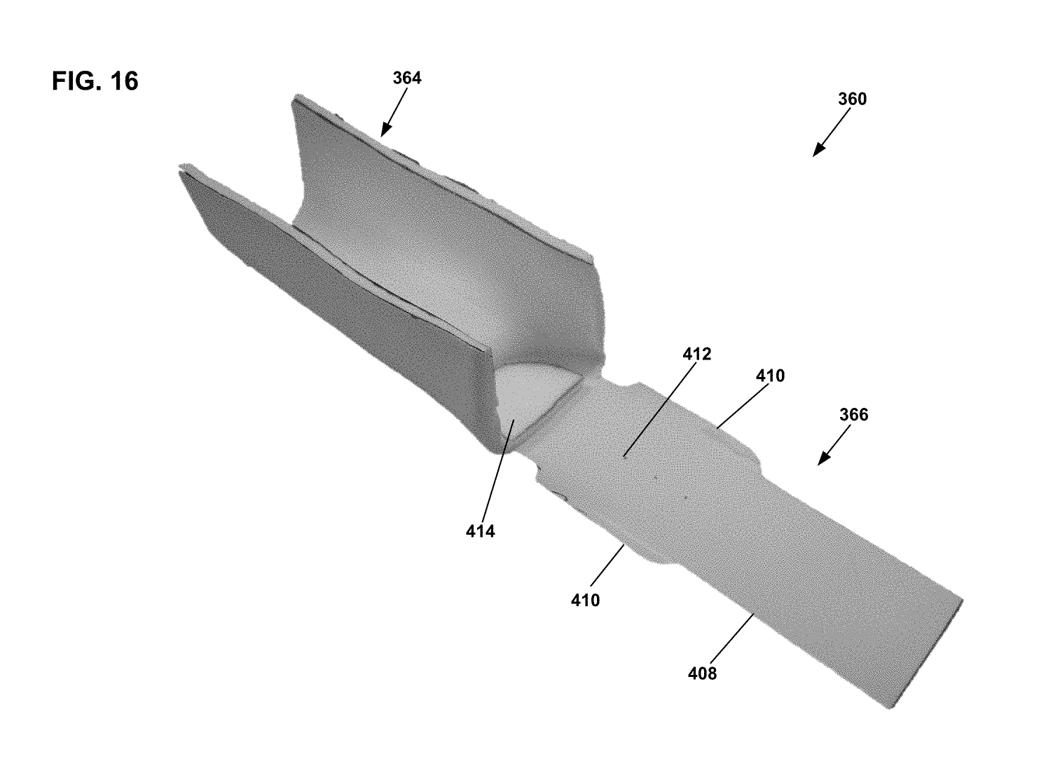

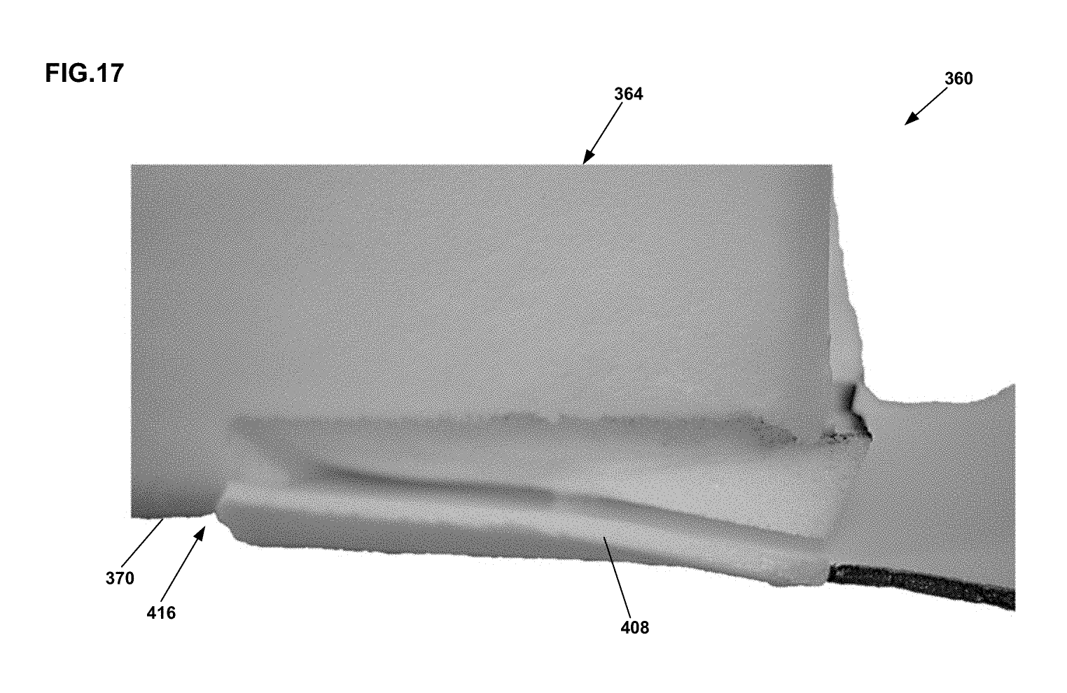

Referring to FIGS. 11-12, various features of the heel float cradle 360 are illustrated. In particular, FIG. 11 is a perspective of an example of a heel float cradle 360. In this example, the heel float cradle 360 includes a lower leg support cradle 364 and a foot support platform 366. In this example, the lower leg support cradle 364 and the foot support platform 366 are connected together to form one continuous piece. The heel float cradle 360 can for example be constructed of a variety of materials including elastomers, polyurethane foam, and/or other open and/or closed cell foams or combinations thereof. The durometer and resilience of the material preferably compresses slightly and springs back to shape upon relieving any pressure. The foot support platform 366 is illustrated and described in more detail with reference to FIGS. 16-17.

In this example, the lower leg support cradle 364 includes a posterior flat surface 370 and side walls 372 that extend along the longitudinal sides of the lower leg. The lower leg support cradle 364 provides a longitudinal channel 368 that receives the lower leg of a patient. The posterior flat surface 370 stabilizes the lower leg support cradle 364 and lower leg against rotation when the patient rests in a supine position and the heel float therapeutic boot 102 is supported on a bed. In this example, the lower leg support cradle 364 has a U-shaped cross-section. In other embodiments, the lower leg support cradle 364 has other cross-sectional shapes, such as, a circular shape.

FIG. 12 is a cross-sectional view of the lower leg support cradle 364 of the heel float cradle 360. As shown, the lower leg support cradle 364 includes an anterior surface 374 along the longitudinal channel 368, a raised surface portion 376, a cutout region 378, and a peripheral edge 380 of the anterior surface 374.

In this example, the anterior surface 374 is contoured and exhibits a compound arcuate shape to support the lower leg or calf. The contoured surface helps to distribute and equalize support on the lower leg or calf and prevents pressure points that might induce skin ulcerations or abrasion. The raised surface portion 376 of the anterior surface 374 is in the region of the Achilles tendon and is shaped to elevate and support the heel in the cutout region 378 with minimal pressure and contact with the boot and away from any support structure, such as a bed, foot stool etc. In this example, the cutout region 378 is an inverted U-shape being adjacent to the peripheral edge of the anterior surface 374 of the lower leg support cradle 364. The cutout region 378 is formed to shelter the suspended heel.

FIG. 13 is a perspective view of the foot support portion 114 of the heel float therapeutic boot 102. In this example, the foot support portion 114 includes a heel tab 382, an overlapping foot member 384, and a toe opening 386. The heel tab 382 and the overlapping foot member 384 are integrally formed and have separate fastening mechanisms. The heel tab 382 can be detached independent of the overlapping foot member 384 either for inspection or ventilation to expose the heel area. The foot support portion 114 has a length that helps to prevent the foot from popping out underneath or over the top of the foot support portion 114.

In this example, the heel tab 382 has a fastening mechanism 344c that connects the heel tab 382 to the loop material 354 vertically positioned along the lower leg support portion 112 of the heel float therapeutic boot 102. The heel tab 382 is arranged and configured on the left and right side of the heel float therapeutic boot 102. The heel tab 382 is also provided with a hook patch 346 similar to those described for the upper and lower overlapping members 330, 332. Accordingly, the description for the hook patch 346 and the loop patch 348 is hereby incorporated by reference in its entirety for the heel tab 382.

The overlapping foot member 384 includes a left side panel 388 extending from a left side wall 390 of the foot support portion 114 and a right side panel 392 extending from a right side wall 394 of the foot support portion 114. In this example, the left and right side panels 388, 392 are integrally formed with the left and right side walls 390, 394 respectively. In some embodiments, the overlapping foot member 384 has moisture wicking material to absorb moisture and ventilate the foot. A variety of soft, moisture absorbent, air permeable open weave or porous materials can be used. The inside of the overlapping foot member 384 that faces the foot is covered with the fleece material 356 similar to the upper and lower overlapping members 330, 332.

The overlapping foot member 384 has a fastening mechanism 344d that connects the left side panel 388 together with the right side panel 392. The overlapping foot member 384 may also be provided with a hook patch 346 and a loop patch 348 as the fastening mechanism 344d similar to those described for the upper and lower overlapping members 330, 332. Accordingly, many of the concepts and features for the upper and lower overlapping members 330, 332 are similar to the overlapping foot member 384. As such, the description for the hook patch 346 and the loop patch 348 is hereby incorporated by reference in their entirety for the overlapping foot member 384. In some embodiments, the overlapping foot member 384 has a taper configuration that covers a top portion of the foot while leaving an open portion adjacent to the lower leg support portion 112. In other embodiments, the overlapping foot member 384 can cover the entire top of the foot.

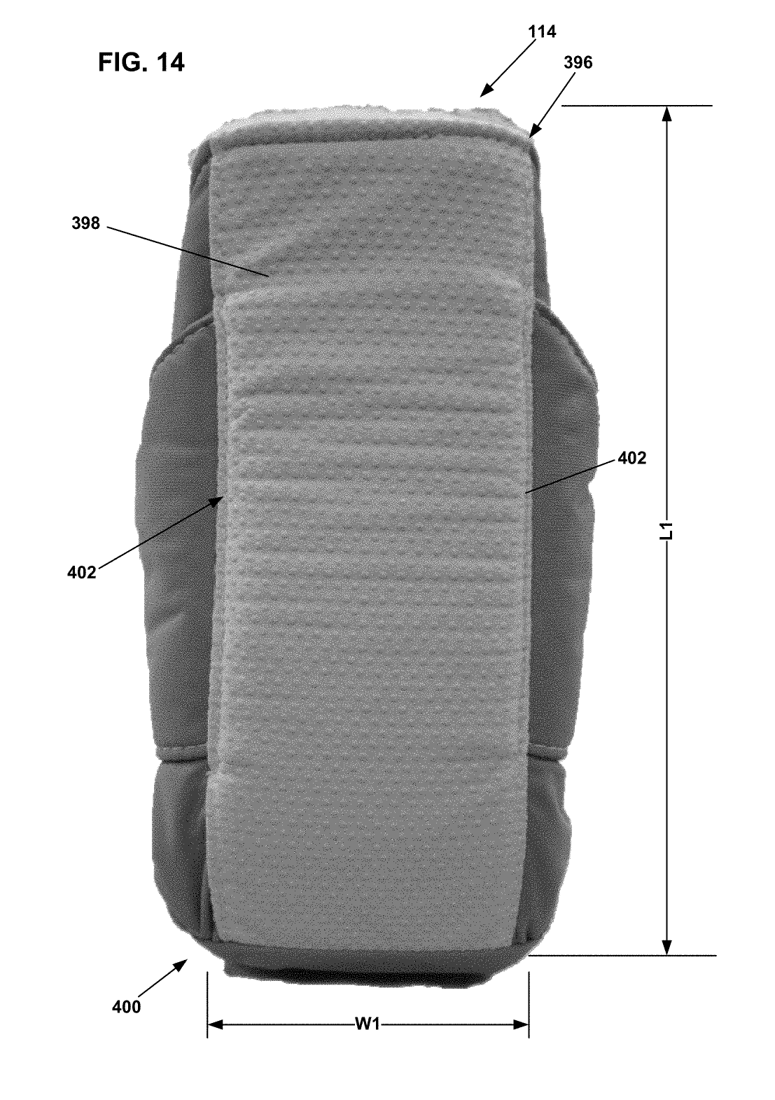

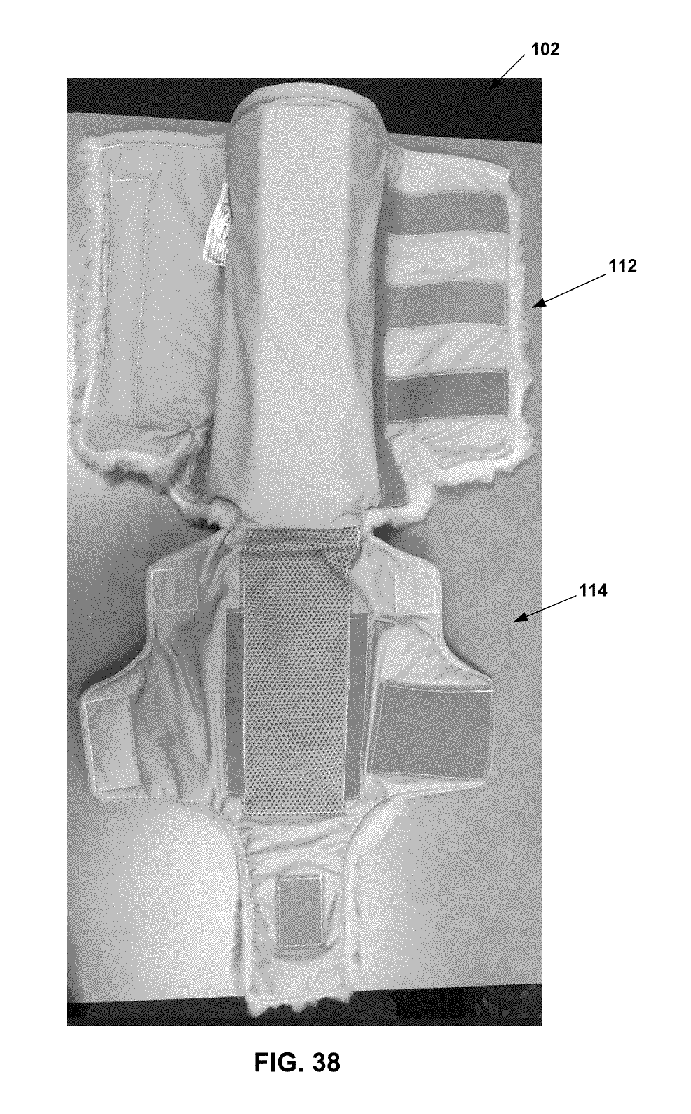

In this example, the toe opening 386 is formed by the overlapping foot member 384. The overlapping foot member 384 covers the toes while still providing for ventilation at the toe opening 386. In other examples, such as one illustrated in FIG. 38, the foot support portion 114 of the heel float therapeutic boot 102 does not provide a toe opening. Instead, a toe portion of the foot support portion 114 can be closed by a toe panel. The toe panel is integrally formed with a base 396 (FIG. 14) and flexes relative to the base 396. In other examples, the toe panel is connected to the base 396 via a fastener, such as a combination of hook and loop fastening materials. Other fasteners can also or alternatively be used, such as buttons, snaps, zippers, adhesive, or other fasteners.

FIG. 14 is a bottom perspective view illustrating exemplary features of the foot support portion 114. In the illustrated example, the foot support portion 114 includes a base 396, a grip material 398, and a flexible hinge 400. The base 396 of the foot support portion 114 has a length L1 and a width W1. In this example, the base 396 of the foot support portion 114 extends beyond the location of the overlapping foot member 384 (FIG. 13) that covers the toes. The base 396 of the foot support portion 114 extends past the toes and acts as a stub which can help to protect the patient while shuffling along a floor.

The grip material 398 covers at least a portion of the base 396 of the foot support portion 114. The grip material 398 acts as a non-slip material to facilitate safe ambulation over smooth tile or wood floors. The grip material 398 can be constructed of a variety of non-slip materials and is shaped to essentially align with and underlie the base 396 of the foot support portion 114. The grip material 398 includes longitudinal sides 402 that extend along the length L1 of the base 396. The grip material 398 is attached to the exterior fabric 362 along the longitudinal sides 402 in the region of the sole. In this example, the grip material 398 is sewn to the exterior fabric 362 of the heel float therapeutic boot 102. Other attachment mechanism may be used, such as, but not limiting to, lamination.

The flexible hinge 400 is formed at the location where the lower leg support portion 112 and the foot support portion 114 are connected together.

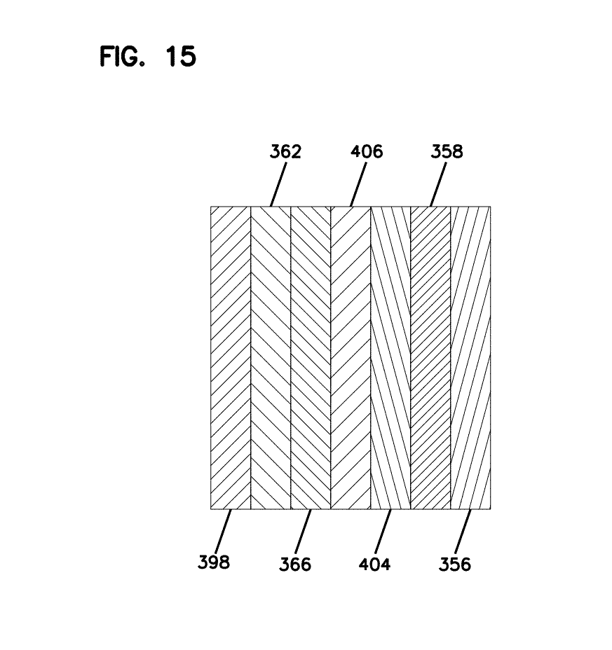

FIG. 15 is a component view of a portion of the foot support portion 114 depicting layers of material therein. In this example, the foot support portion 114 includes a fleece material 356, a lining 358, a foam layer 404, an interior fabric 406, the foot support platform 366, the exterior fabric 362, and the grip material 398. As noted above, the foot support platform 366 is illustrated and described in more detail with reference to FIG. 16. The construction of multiple layers provides a more firm platform for the foot to rest against. The foam layer 404 provides for more cushion to the foot and reduces pressure thereon. The interior fabric 406 is of a type similar to the exterior fabric 362 but rather lined inside portions of the heel float therapeutic boot 102. Many of the concepts and features for these layers have been disclosed or are similar to the lower leg support portion 112 shown in FIG. 10. Accordingly, the descriptions of these layers are hereby incorporated by reference in their entirety for the foot support portion 114.

Referring to FIGS. 16-17, various features of the foot support platform 366 of the heel float cradle 360 are illustrated. In particular, FIG. 16 is a perspective view of the foot support platform 366 of the heel float cradle 360. In this example, the foot support platform 366 includes an extended flap portion 408, flaps 410, apertures 412, and foam 414.

The extended flap portion 408 is arranged and configured to align with the base 396 of the foot support portion 114. In this example, the extended flap portion 408 is constructed of closed cell foam, approximately ⅜-inch thick. In other embodiments, the thickness of the extended flap portion 408 can be greater or less. A proximal end of the extended flap portion 408 is bonded to a recess 416 (depicted in FIG. 17) formed into the posterior flat surface 370 of the lower leg support cradle 364 adjacent an inverted U-shaped cutout region 378 being adjacent to the peripheral edge of the anterior surface 374 of the lower leg support cradle 364. The lower leg support cradle 364 and the foot support platform 366 are married together at the recess 416 to create the hinge 400 point in the heel float therapeutic boot 102. The extended flap portion 408 includes apertures 412 to aerate the foot.

The flaps 410 extend from opposite sides of the extended flap portion 408. The flaps 410 are joined with the other layers in the foot support portion 114. In this example, the wing support structure 306 leverages support from the flaps 410 while being adjustably fastened as desired. The wing support structure 306 is illustrated and described in more detail with reference to FIGS. 19-21. The layers of the foot support portion 114 are attached together along the flaps 410 in the region of the sole. In this example, the layers are sewn together through the flaps 410.

The foam 414 is positioned on the extended flap portion 408 at the proximal end below the raised heel. The foam 414 is below the cutout region 378 and provides the cushion for the patient's heel. In this example, the foam 414 is shaped as a half moon. In other embodiments, the foam 414 can take the form of other shapes, such as, circles, square, or rectangular etc.

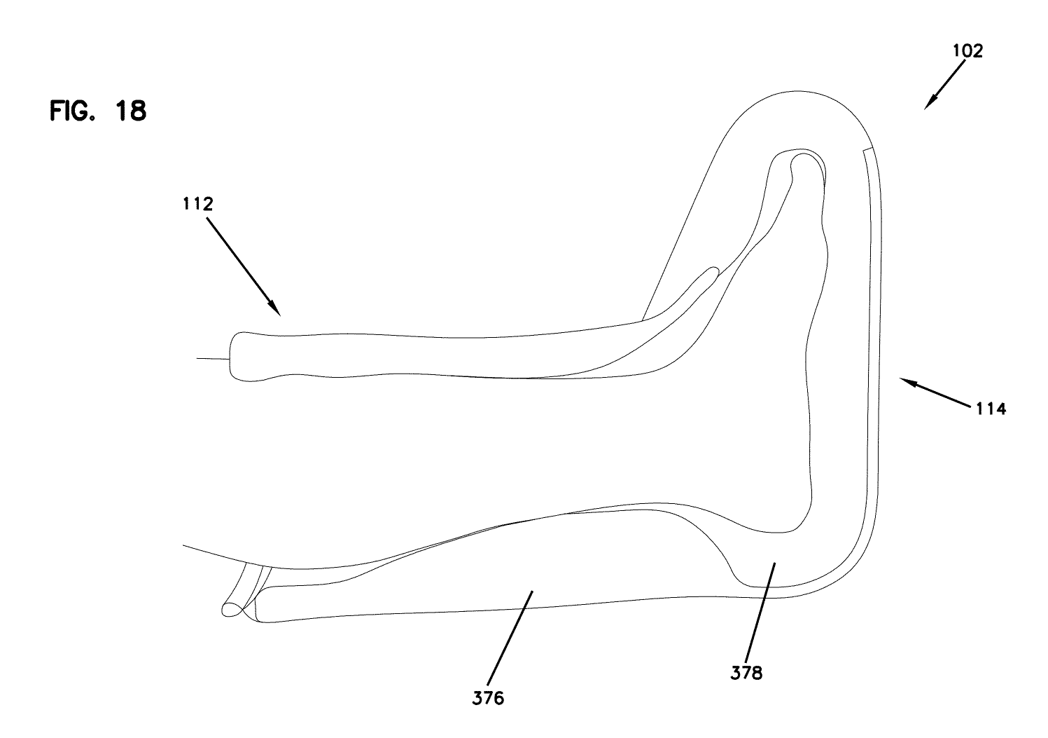

FIG. 18 depicts a cross-sectional view of an example of a patient's foot in the heel float therapeutic boot 102. In this example, the heel is shown elevated with the foot positioned upright at approximately 90 degrees.

FIG. 19 is a side perspective view of the heel float therapeutic boot 102. In this example, the wing support structure 306 is attached to a heel float therapeutic boot 102.

As described above, the boot 102 includes one or more elements that are included in the fastening device 160 configured to removably mount the support device 104 to the boot 102. In some embodiments, the boot 102 includes a boot fastener 164 corresponding to the support device fastener 162 formed on the support device 104.

The boot fastener 164 is configured to be complementary to the support device fastener 162. In the illustrated example in which the support device fastener 162 includes a first support device fastening patch 166 and a second support device fastening patch 168, the boot fastener 164 includes a first boot fastening patch 430 and a second boot fastening patch 432.

When the support device 104 is in the first position as illustrated in FIG. 4, at least a portion of the first support device fastening patch 166 is attached to at least a portion of the first boot fastening patch 430, and at least a portion of the second support device fastening patch 168 is attached to at least a portion of the second boot fastening patch 432. When the support device 104 is in the second position as illustrated in FIG. 5, at least a portion of the first support device fastening patch 166 is attached to at least a portion of the second boot fastening patch 432, and at least a portion of the second support device fastening patch 168 is attached to at least a portion of the first boot fastening patch 430.

The first boot fastening patch 430 is arranged either or both of the side walls 126, 128 of the lower leg support portion 112 of the boot 102. In some embodiments, the first boot fastening patch 430 extends along at least a portion of the length of the lower leg support portion 112 so that the support device 104 can be mounted to any longitudinal location along the lower leg support portion 112.

The second boot fastening patch 432 is arranged either or both of the sides walls 126, 128 of the lower leg support portion 112 of the boot 102. In some embodiments, the second boot fastening patch 432 is arranged generally in parallel with the first boot fastening patch 430.

In some embodiments, the second boot fastening patch 432 can be formed on the wing support structure 306 so that the support device 104 is mounted onto the wing support structure 306. In addition or alternatively, a portion of the leg support attachment element 490, such as the loop material 354, can be used as the second boot fastening patch 432. In the illustrated example, a portion of the leg support attachment element 490 is not covered by the wing support structure 306 and thus can be used to mount one of the first support device fastening patch 166 and the second support device fastening patch 168. In this configuration, the support device 104 can be selectively located at any location away from the foot support portion 114 (or close to an end of the lower leg support portion 112 opposite to the foot support portion 114).

Referring still to FIG. 19, the wing support structure 306 of the heel float therapeutic boot 102 supports the foot in a preferred position. The wing support structure 306 is attached to the heel float therapeutic boot 102 and is configured to be adjustable to obtain the preferred position. The arrangement and configuration of the back side 428 of the wing support structure 306 provides for flexibility of attaching the wing support structure 306 to the loop material 354 that helps place the foot support portion 114 in the proper position. The wing support structure 306 can be placed anywhere along the loop material 354 as desired. The positioning of the wing support structure 306 will vary with each patient. By way of example, a proper fit can be obtained for any patient from a pediatric size foot up to a man's size 34 foot. The heel float therapeutic boot with the wing support structure can also be configured to support other sizes of foot. The configuration of the wing support structure 306 also provides for easy access to the heel of the foot.

The wing support structure 306 is adjustable to be positioned along the loop material 354. In this example, the wing support structure 306 is adjustable along the lower leg support portion 112 perpendicular to Axis A. Axis A extends longitudinally through the foot support portion 114. The wing support structure 306 has the flexibility to position the foot between 75-110 degrees about axis A. This feature provides for a patient to have the ability to access any part of the bottom of the foot and position the foot as desired. The width W2 of the bottom side 42 of the wing support structure 306 and the configuration of attachment not only helps to keep the foot in an upright position, but it also helps to keep the foot in place so that it is less likely to rotate or migrate around inside of the heel float therapeutic boot 102.

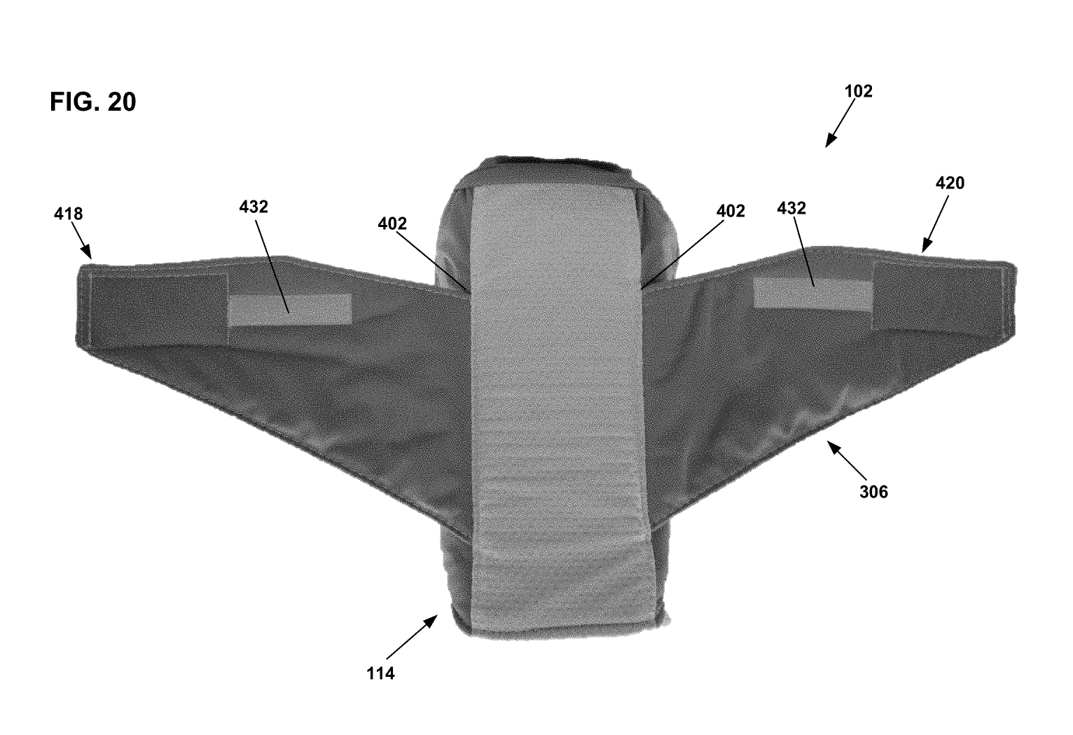

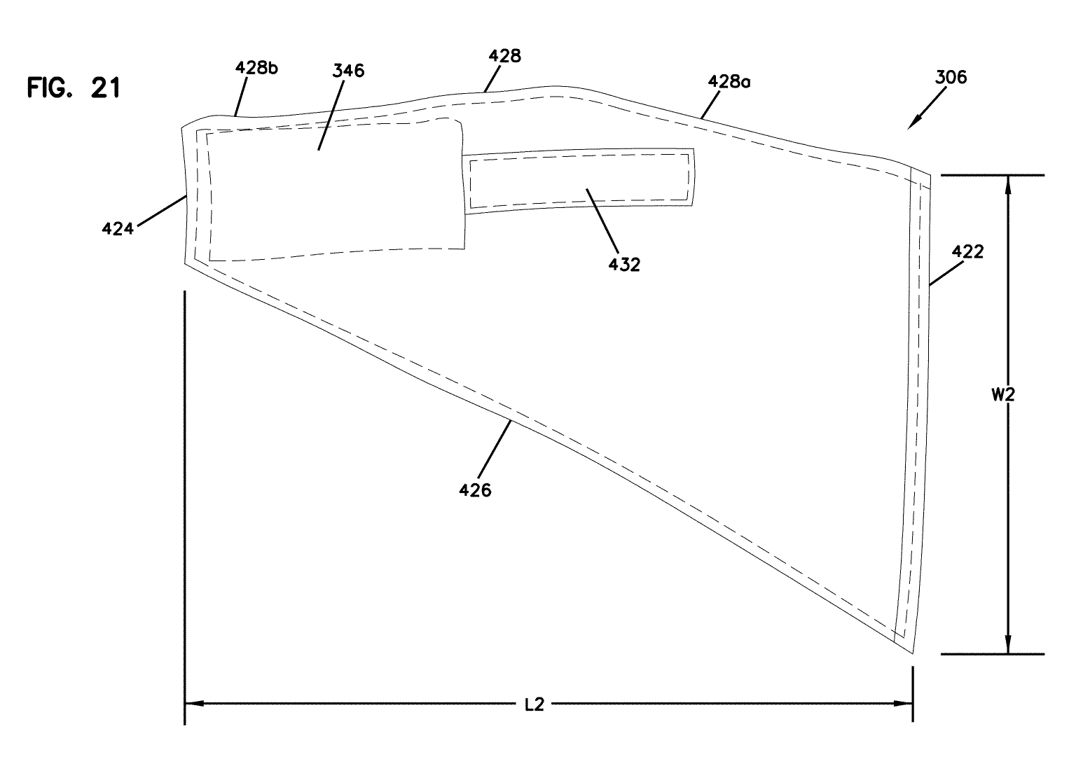

Referring to FIGS. 20-21, various features of the wing support structure 306 are illustrated. In particular, FIGS. 20-21 are perspective views of the wing support structure 306. Referring to FIG. 20, the bottom side 422 of the wing support structure 306 is sewn along the longitudinal sides 402 and intersects together with the layers in the foot support portion 114. Portions of the wing support structure 306 extend beyond the sewn area along the longitudinal sides 402 to help prevent the wing support structure 306 from detaching from the foot support portion 114. The bottom side 422 of the wing support structure 306 has a width W2 that expands along a portion of the base 396 of the foot support portion 114. The bottom side 422 of the wing support structure 306 is positioned mostly centered on the base 396. The configuration of the wing support structure 306 eliminates the problem of generating pressure points underneath the foot because no portion of the wing support structure 306 goes across the ball of the foot. The wing support structure 306 is anchored to the sole and helps to spread the pressure points or force across the patient's foot while holding the foot support portion 114 of the heel float therapeutic boot 102 in a desired position. The wing support structure 306 is arranged and configured to keep the foot support portion 114 of the heel float therapeutic boot 102 pulled up or upright.

In the illustrated example, the wing support structure 306 includes a left support wing 418 and a right support wing 420. The left and right support wings 418, 420 are arranged and configured on opposite sides of the heel float therapeutic boot 102. The left and right support wings 418, 420 are each attached to the heel float therapeutic boot 102 along the longitudinal sides 402 in the region of the sole. In the illustrated example, the left and right support wings 418, 420 are permanently fastened to the foot support portion 114. The configuration provides for a wing support structure 306 that is non-adjustable in relation to the foot support portion 114. The wing support structure 306 is adjustably fastenable along the sides of the lower leg support portion 112 (shown in FIG. 19). The wing support structure 306 is wide enough and fixed to the foot support portion 114 to help prevent the foot of a patient from wrapping across the top of the heel float therapeutic boot 102 or popping out underneath the heel float therapeutic boot 102. The wing support structure 306 has a wide area to help disperse any points of pressure, which lowers the likelihood of having a pressure area anywhere in the foot region. Having the wing support structure 306 permanently attached at the sole not only helps to prevent pressure points on the foot but it also helps to eliminate improper positioning of the strap over the foot.

Referring to FIG. 21, the wing support structure 306 includes a bottom side 422, an opposing top side 424, a front side 426, a back side 428, and the second boot fastening patch 432. The wing support structure 306 further includes a length L2 and a width W2.

In the illustrated example, the front side 426 of the wing support structure 306 is angled relative to the back side 428 of the wing support structure 306 such that the top side 424 of the wing support structure 306 is narrower than the bottom side 422 of the wing support structure 306. The front side 426 of the wing support structure 306 being about 9 to 31 inches in length. The top side 424 of the wing support structure 306 includes a hook patch 346 as already described above. The top side 424 is attached to the loop material 354 to position the wing support structure 306 as desired. The top side 424 of the wing support structure 306 being about 3 to 4 inches in length.

In the illustrated example, the back side 428 of the wing support structure 306 is about 9 to 30 inches in length. In some examples, the back side 428 is streamlined. For example, the back side 428 has an angled portion 428a parallel or generally parallel to the front side 426 of the wing support structure 306. Such an angled portion helps prevent the wing support structure 306 from being caught by an object, such as a portion of a wheelchair or a bed, such as by reducing excess material and also by offsetting the wing support structure 426 from the forward and rearward ends of the foot support structure. The angled portion 428a is about 4 to 5 inches in length. The angled portion 428a extends from the bottom side 422 of the wing support structure 306. The angled portion 428a forms a perpendicular portion 428b that is perpendicular to the bottom side 422 of the wing support structure 306. The perpendicular portion 428b being about 4 to 5 inches in length. In other embodiments, other shapes and configurations are possible. The left and right support wings 418, 420 are shaped expanding a length and width with respect to the heel float therapeutic boot 102. In other embodiments, other shapes, sizes, and configurations are possible.

The left and right support wings 418, 420 each include moisture wicking material similar to the overlapping foot member 384. The left and right support wings 418, 420 each have a fastening mechanism 344e. As illustrated in FIG. 19, the left and right support wings 418, 420 are provided with the hook patch 346 and the loop material 354 as the fastening mechanism 344e similar to those described above. As such, the description for the hook patch 346 and the loop material 354 is hereby incorporated by reference in their entirety for the left and right support wings 418, 420.

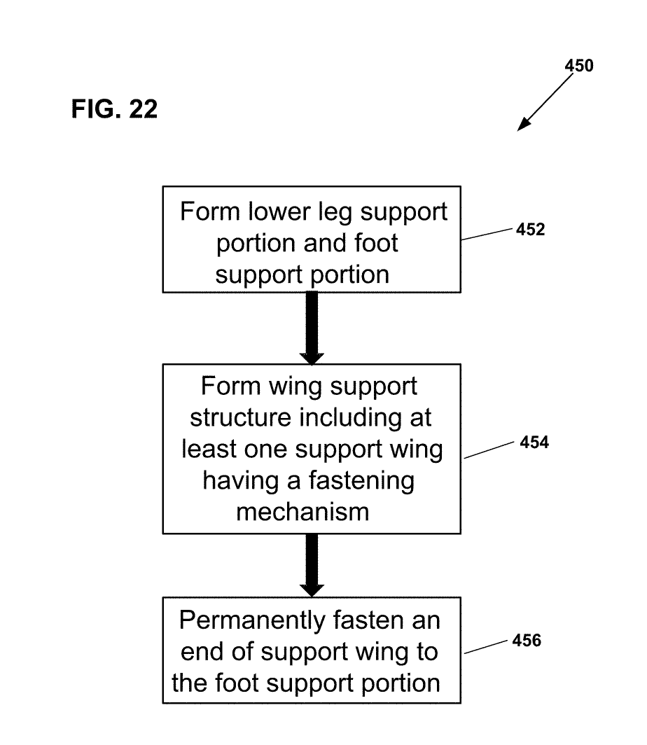

FIG. 22 is a flow chart illustrating an example method 450 of making a boot 102. In this example, the method 450 includes operations 452, 454, and 456.

The operation 452 is performed to form a lower leg support portion 112 and a foot support portion 114. Examples of the lower leg support portion 112 and the foot support portion 114 are shown and described with reference to FIGS. 6-8. In some embodiments, the foot support portion 114 includes a foot support platform 366. An example of a foot support platform 366 is shown and described with reference to FIG. 16.

The operation 454 is performed to form the wing support structure 306. In some embodiments, the wing support structure 306 includes at least one support wing 306(A,B). In some embodiments, the wing support structure includes a left support wing 306A and a right support wing 306B. The support wings 306 include a first end portion and a second end portion. In some examples, the first end portion includes the bottom side 422 of the support wings, and the second end portion includes the top side 424 and/or the back side 428 of the support wings. The second end portion is configured to be adjustably fastened to opposite sides of the lower leg support portion. Examples of the wing support structure 306 and support wings 306A and 306B are illustrated and described in more detail in FIGS. 19-21.

The operation 456 is performed to permanently fasten the first end portion of the support wing 306(A,B) to the foot support portion 114. In some embodiments, operation 456 includes permanently fastening the first end portions of the left and right support wings 306A and 306B to the foot support portion 114. In some embodiments, the one or more support wings 306 are permanently fastened to the foot support platform 366. An example of the fastening operation is sewing with thread. Other fastening operations can also be used, such as described herein. Examples showing the attachment of the wing support structure 306 and support wings 306A and 306B to the foot support portion 114 are shown in FIGS. 6 and 19.

After the boot 102 has been made, a method of using the boot 102 can be performed. In one example embodiment, the boot 102 is arranged on a foot and lower leg of a patient, such as illustrated in FIG. 18. The foot and lower leg are then secured in the boot 102 using the various panels and fasteners described herein. Additionally, the second end of one or more of the support wings 306A and 306B are adjustably secured to sides of the lower leg support portion 112. The boot 102 securely supports the foot and lower leg in a desired position.

Referring to FIGS. 23-42, another example embodiment of the heel float therapeutic boot 102 is described. As many of the concepts and features are similar to the first embodiment shown in FIGS. 6-22, the description for the first embodiment is hereby incorporated by reference for this embodiment. Where like or similar features or elements are shown, the same reference numbers are used where possible. The following description of this example embodiment will be focused primarily to the differences from the first embodiment.

In this embodiment, the wing support structure 306 is configured to be removably attached to the therapeutic boot 102. Similar to the embodiments in FIGS. 6-22, the wing support structure 306 is also adjustably attached to the therapeutic boot 102 to support the foot in different positions.

Further, the boot 102 of this embodiment does not include an opening, such as the opening 350, in the lower leg support portion 112. Rather, the lower leg support portion is configured such that a single overlapping member wraps a lower leg portion in a closed configuration. In addition, the boot 102 does not provide a toe opening, such as the toe opening 386, in the foot support portion 114. As described below, the boot 102 includes a toe cover panel 387 that extends from the base 396 of the foot support portion 114 and flexes toward a space defined by the foot support portion 114 to close the space. In this configuration, the overlapping foot member 384 and the toe cover panel 387 completely cover the foot including the toe. However, other embodiments can include an opening.

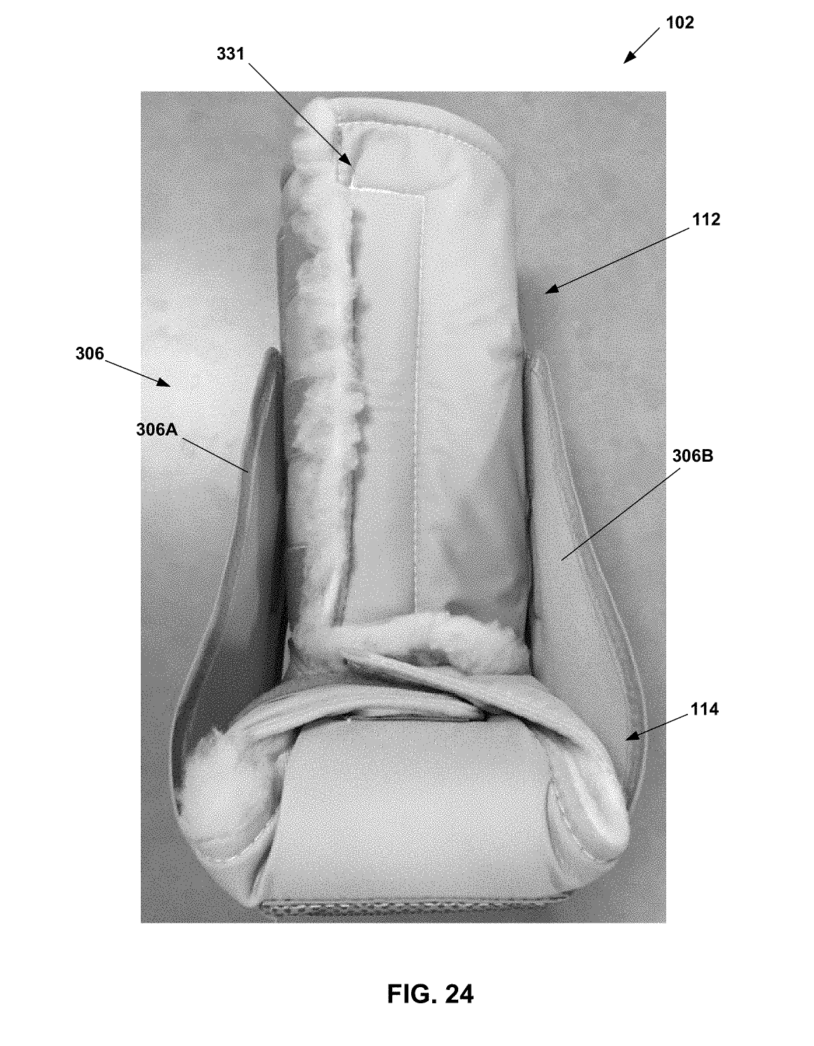

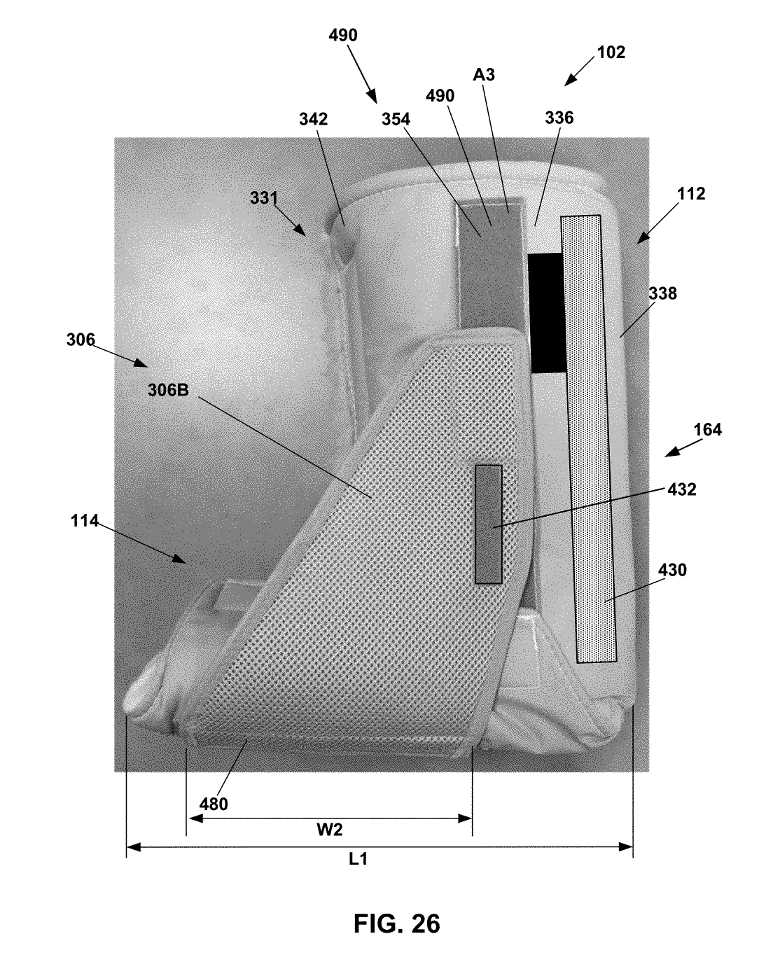

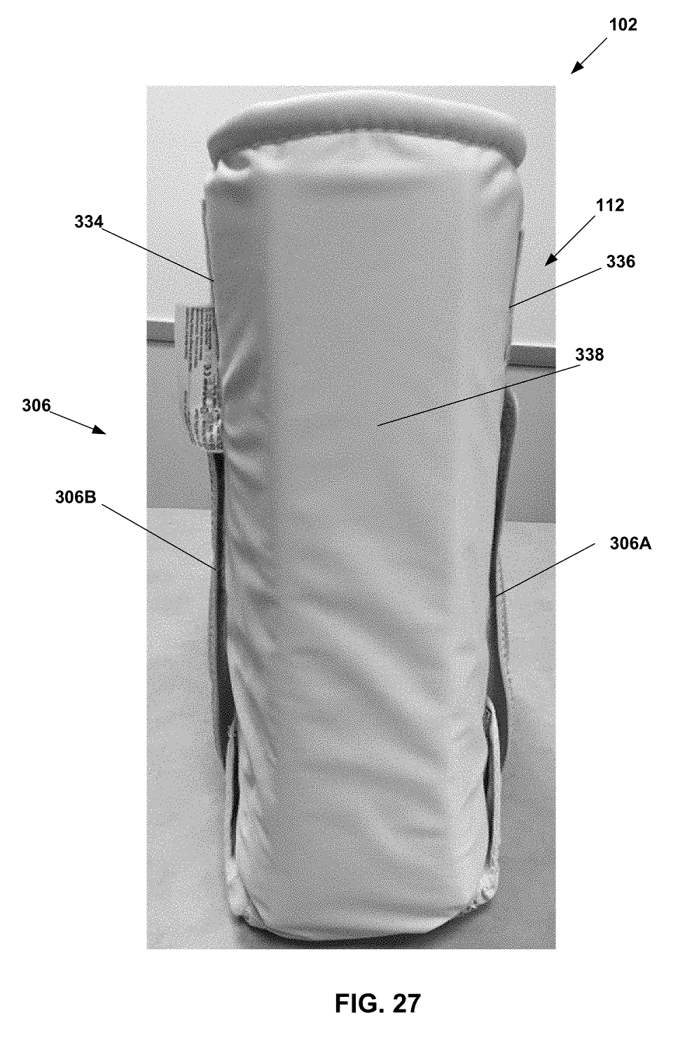

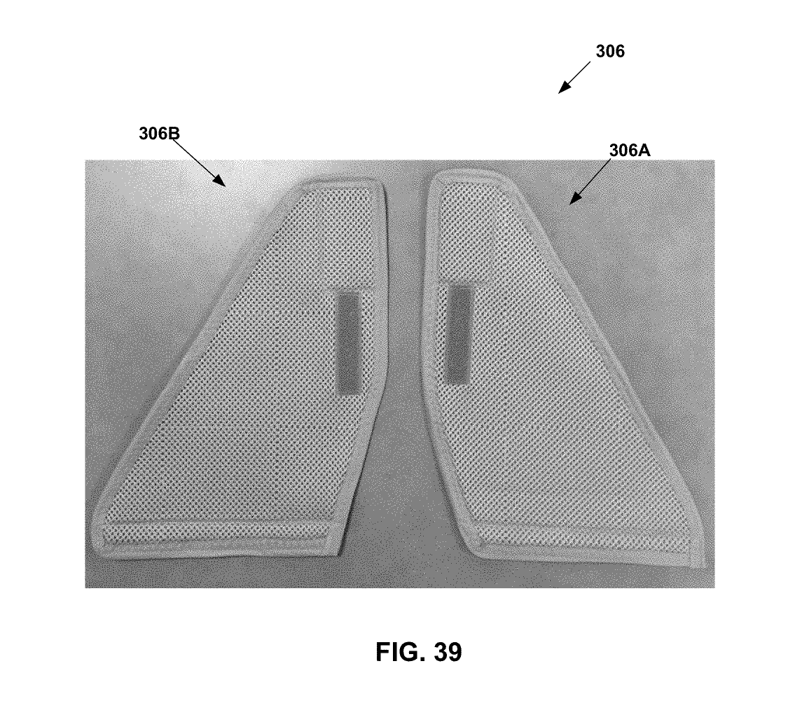

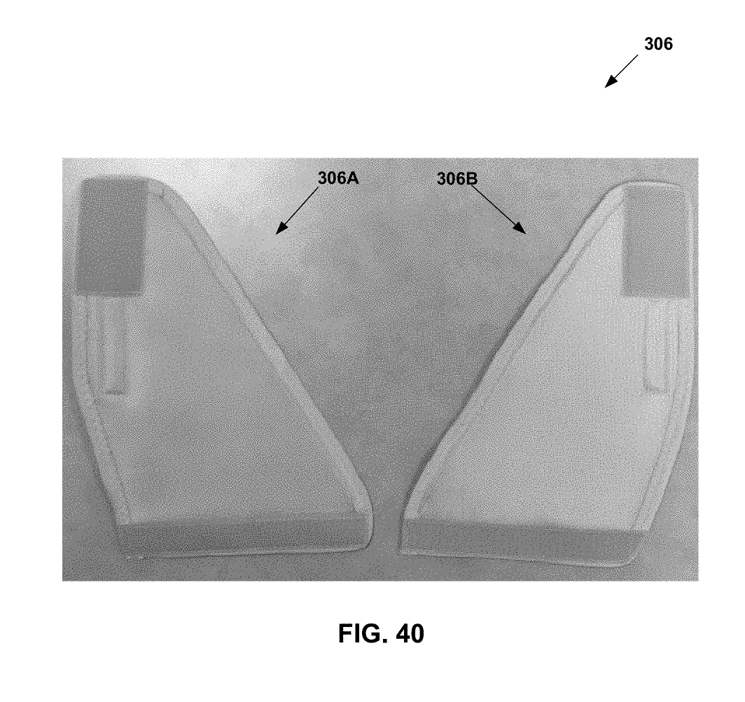

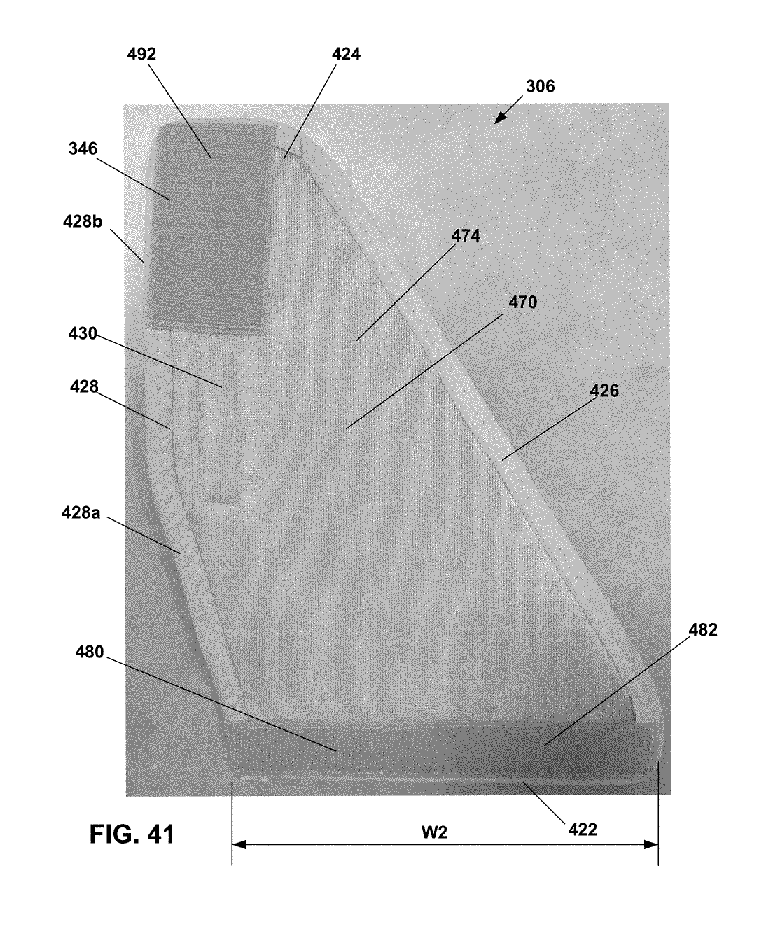

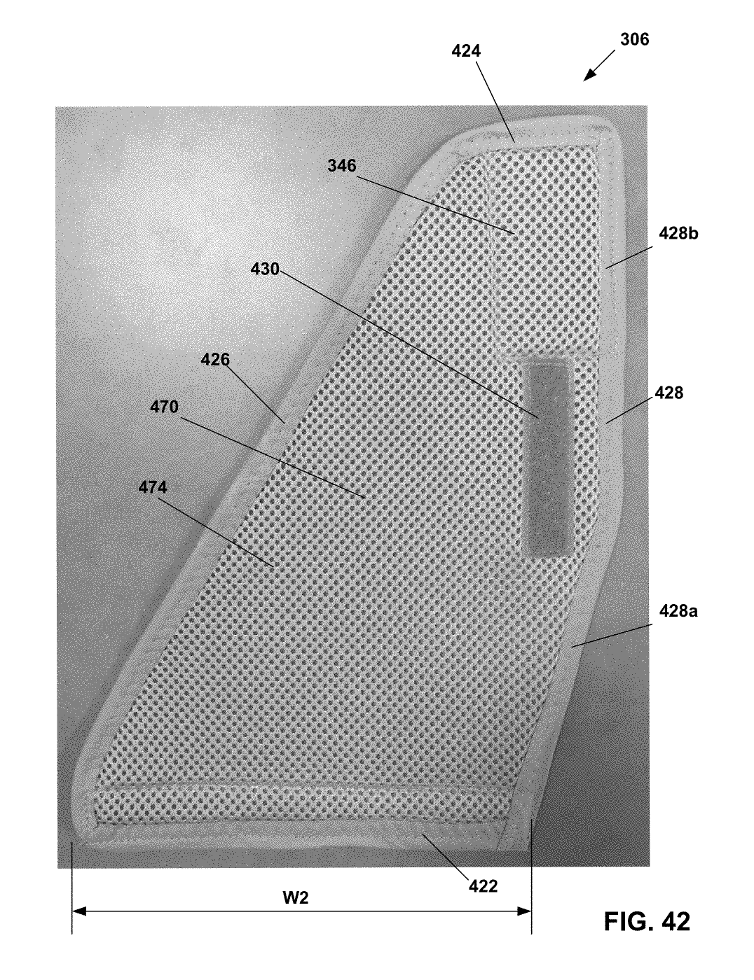

Referring to FIGS. 23-27 and 39-42, another example of the wing support structure (also referred to herein as a foot support device or side support device) is further described. In particular, FIG. 23 is a perspective view of another example of the heel float therapeutic boot 102. FIG. 24 is a front view of the heel float therapeutic boot 102 of FIG. 23. FIG. 25 is a first side view (e.g., a left side view) of the heel float therapeutic boot 102 of FIG. 23, and FIG. 26 is a second side view (e.g., a right side view) of the heel float therapeutic boot 102 of FIG. 23. FIG. 27 is a rear view of the heel float therapeutic boot 102 of FIG. 23. FIG. 39 is a front view of an example set of foot support device. FIG. 40 is a rear view of the set of foot support device of FIG. 39. FIG. 41 is a front view of the foot support device of FIG. 39. FIG. 42 is a rear view of the foot support device of FIG. 41. In this document, the wing support structure 306 is also referred to as a foot support device or structure or a side support device or structure to reflect the functional aspect of the wing support structure.

In this embodiment, the wing support structure 306 is configured similarly to the wing support structure 306 in FIGS. 19-21. The wing support structure 306 includes a body 470 having an inner surface 472 and an outer surface 474 opposite to the inner surface 472. When attached to the boot 102, the wing support structure 306 is arranged such that the inner surface 472 of the body 470 is oriented inwardly relative to the boot 102 and the outer surface 474 of the body 470 faces outwardly relative to the boot 102.

The wing support structure 306 has the bottom side 422, the top side 424, the front side 426, and the back side 428, similarly to the wing support structure of FIGS. 19-21.

On the inner surface 472 of the body 470 is provided a first attachment element 480 configured to removably attach the wing support structure 306 to the boot 102 at a first end portion of the wing support structure 306. In some examples, the first attachment element 480 is arranged at the bottom side 428 of the wing support structure 306 and configured to be detachably engaged with the foot support portion 114 of the boot 102.

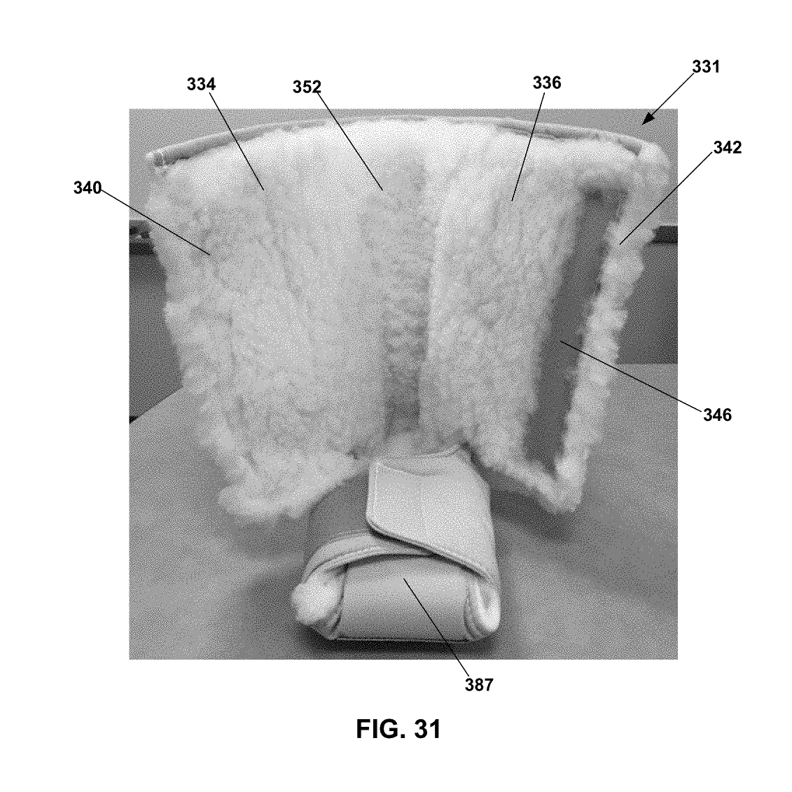

In some examples, the first attachment element 480 is arranged along the entire width W2 of the bottom side 428 of the body 470, as illustrated in FIG. 31. In this configuration, the width W2 of the wing support structure 306 at the bottom side 428 is substantially the same as the length of the first attachment element 480. In other examples, the first attachment element 480 extends only a portion of the width W2 at the bottom side 428.

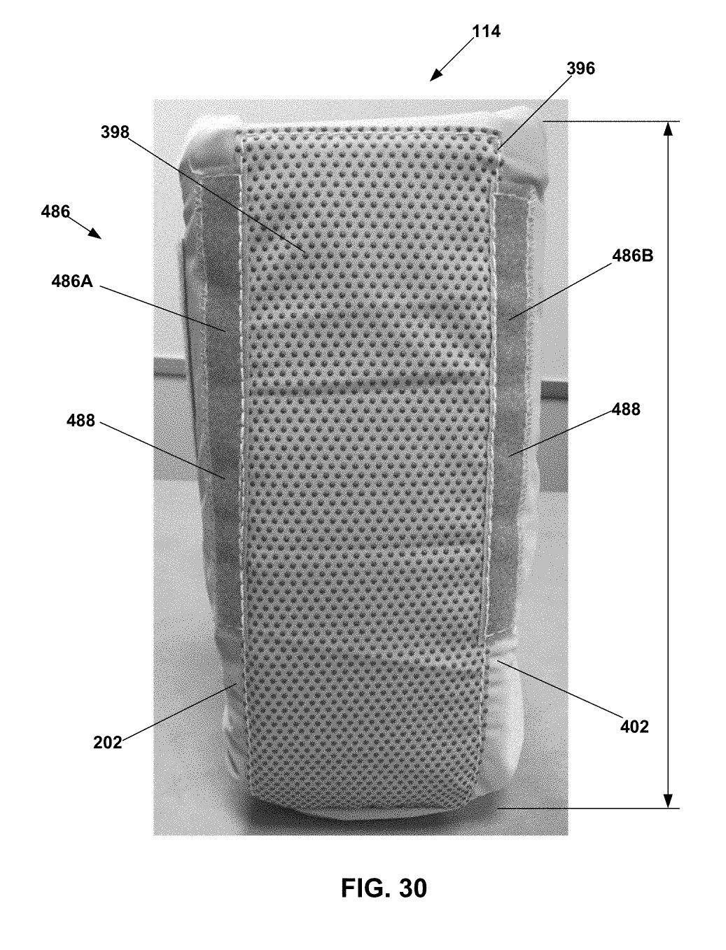

The first attachment element 480 can be of various types. In the illustrated example, the first attachment element 480 includes a hook patch 482 when a corresponding attachment element (e.g., a foot support attachment element 486) on the foot support portion 114 includes a loop patch 488 (FIG. 30). One example of such a combination of the hook patch and the loop patch in this document is available from Velcro Companies, United Kingdom. Other types, such as buttons, adhesive, hooks, clips, clasps, bolts, straps, or combinations of these or other fastening mechanisms.

Referring to FIG. 30, as described herein, the foot support portion 114 includes the base 396 that is designed to extend along the sole of the foot when the boot 102 covers the leg. The base 396 of the foot support portion 114 includes a foot support attachment element 486 configured to removably attach the first attachment element 480 of the wing support structure 306. In the illustrated example, the foot support attachment element 486 includes a first foot support attachment element 486A and a second foot support attachment element 486B, which are configured to removably fasten the first attachment elements 480 of the first and second (i.e., the right and left) support wings 306A and 306B, respectively.

In some embodiments, the foot support attachment element 486 includes a loop patch 488, which is complementary to the hook patch 482 of the first attachment element 480 of the wing support structure 306.

In some examples, the foot support attachment elements 486 are arranged along the longitudinal sides 402 of the base 396. For example, the foot support attachment elements 486 are positioned at or adjacent the edges, at which the base 396 and the right and left side walls 390, 394 meet, along the longitudinal sides 402 of the base 396. The foot support attachment elements 486 are positioned closer a toe end of the foot support portion 114 than a heel end of the foot support portion 114. In some embodiment, the foot support attachment elements 486 are positioned between a longitudinal axis A3 (FIG. 25) defined along the length of the lower leg support portion 112 and the toe end of the foot support portion 114. In some embodiments, the longitudinal axis A3 is arranged substantially in the middle of the width of the lower leg support portion 112 as seen in FIG. 25. In other embodiments, the longitudinal axis A3 is substantially aligned with a leg support attachment element 490 (e.g., the loop material 354) of the lower leg support portion 112. By arranging the foot support attachment elements 486 closer to the toe end of the foot support portion 114, the bottom side 422 of the wing support structure 306 are fixed to be positioned adjacent the plantar fascia, rather than calcaneus (heel bone) of the foot.

The length W2 of the first attachment element 480 arranged along the bottom side 422 can vary. The length W2 of the first attachment element 480 is sized to engage a majority of the length of the base 396 so that the pressure points or force on the patent's foot are effectively spread across the foot. In some examples, the length W2 of the first attachment element 480 of the wing support structure 306 is larger than a length of the top side 424. In some examples, the length W2 of the first attachment element 480 is greater than about a half of the length L1 of the foot support portion 114. In other examples, the length W2 of the first attachment element 480 is greater than about 70% of the length L1 of the foot support portion 114. As described above, in some embodiments, the bottom side 422 of the wing support structure 306 is located between the longitudinal axis A3 (FIG. 25) and the toe end of the foot support portion 114, so that the pulling force is spread across the plantar fascia portion, not the heel portion of the foot.

Referring again to FIG. 41, the wing support structure 306 includes a second attachment element 492 on the inner surface 472 of the body 470. The second attachment element 492 is configured to removably attach the wing support structure 306 to the boot 102 at a second end portion of the wing support structure 306. In some examples, the second attachment element 492 is arranged at the back side 428 of the wing support structure 306 and configured to be detachably engaged with the lower leg support portion 112 of the boot 102. In some examples, the second attachment element 492 is arranged along at least a portion of the back side 428 of the wing support structure 306, as illustrated in FIG. 41.

The second attachment element 492 can be of various types. In the illustrated example, the second attachment element 492 includes the hook patch 346 where the corresponding leg support attachment element 490 includes the loop material 354. The arrangement and configuration of the second attachment element 492 is similar to the arrangement and configuration of the loop material 354 as described herein.

Referring to FIGS. 25-29, as described herein, the lower leg support portion 112 includes the leg support attachment element 490, such as the loop material or patch 354 as described herein. In this embodiment, the lower leg support portion 112 extends along the length of the side walls 334, 336 so that the second attachment element 492 of the wing support structure 306 is adjustably fastened thereon.

Similarly to the boot 102 as illustrated in FIG. 19, the boot 102 as illustrated in FIGS. 23-42 includes one or more elements included in the fastening device 160. In some embodiments, the boot 102 includes a boot fastener 164 corresponding to the support device fastener 162 formed on the support device 104. The boot fastener 164 in this example is similarly configured as the boot fastener 164 in FIG. 19. Therefore, the description of the boot fastener 164 in this example is omitted for brevity purposes.

In some examples, the body of the wing support structure of the present disclosure is made of one or more breathable material. In addition, or alternatively, the body of the wing support structure of the present disclosure is made of one or more breathable material is made of one or more stretchable material.

The various examples and teachings described above are provided by way of illustration only and should not be construed to limit the scope of the present disclosure. Those skilled in the art will readily recognize various modifications and changes that may be made without following the example embodiments and applications illustrated and described herein, and without departing from the true spirit and scope of the present disclosure.

What is claimed is:

1. A heel float therapeutic footwear apparatus comprising:
a boot including:
a lower leg support portion configured to at least partially wrap a lower leg, the lower leg support portion having a front wall, a back wall, and opposite side walls, wherein the front wall at least partially surrounds a front portion of the lower leg, the back wall at least partially surrounds a back portion of the lower leg, and the side walls at least partially surrounds sides of the lower leg; and
a foot support portion extending from the lower leg support portion; and
a lateral support device configured to laterally support the lower leg support portion, the lateral support device having a mounting face abutted to one of the side walls, the mounting face having a curved surface complementary to a curved shape of the side walls.

2. The apparatus of claim 1, wherein the lateral support device is detachably mounted to the one of the side walls.

3. The apparatus of claim 1, wherein the lateral support device has a first base face abutted to the mounting face at a first edge, and a second base face abutted to the mounting face at a second edge, the first base face abutted to the second base face at a third edge and arranged at an first angle relative to the second base face, the first angle being an acute angle.

4. The apparatus of claim 1, wherein the angle ranges between 30 degrees and 60 degrees.

5. The apparatus of claim 1, wherein the first base face forms a second angle relative to the mounting face at the first edge, and the second base face forms a third angle relative to the mounting face at the second edge, the second angle different from the third angle.

6. The apparatus of claim 1, wherein the second angle ranges between 20 degrees and 80 degrees, and the third angle ranges between 20 degrees and 80 degrees.

7. The apparatus of claim 3, wherein the lateral support device is selectively arranged in a first orientation or a second orientation, the first orientation in which the first base face is positioned as a bottom of the lateral support device and the mounting face is abutted to the one of the side walls and supports the boot at a first boot angle relative to the bottom, and the second orientation in which the second base face is positioned as the bottom of the lateral support device and the mounting face is abutted to the one of the side walls and supports the boot at a second boot angle relative to the bottom.

8. The apparatus of claim 7, wherein the first boot angle ranges between 60 degrees and 120 degrees, and the second boot angle ranges between 30 degrees and 90 degrees.

9. The apparatus of claim 3, wherein the lateral support device is configured such that:
when the lateral support device is arranged such that the first base face is positioned as a bottom of the lateral support device, a first imaginary plane orthogonal to the bottom and including the second edge is positioned between a second imaginary plane orthogonal to the bottom and including the first edge of the mounting face and a third imaginary plane orthogonal to the bottom and including the third edge; and
when the lateral support device is arranged such that the second base face is positioned as the bottom of the lateral support device, a fourth imaginary plane orthogonal to the bottom and including the first edge is positioned between a fifth imaginary plane orthogonal to the bottom and including the second edge and a sixth imaginary plane orthogonal to the bottom and including the third edge.

10. The apparatus of claim 9, wherein the first imaginary plane and the second imaginary plane are spaced apart at a first distance, and the first imaginary plane and the third imaginary plane are spaced apart at a second distance, and wherein a ratio of the first distance to the second distance ranges 1:2 and 1:5.

11. The apparatus of claim 9, wherein the forth imaginary plane and the fifth imaginary plane are spaced apart at a third distance, and the fourth imaginary plane and the sixth imaginary plane are spaced apart at a fourth distance, and wherein a ratio of the third distance to the fourth distance ranges 1:1.3 and 1:2.5.

12. The apparatus of claim 1, further comprising a fastening device configured to detachably mount the mounting face of the lateral support device to the one of the side walls.

13. The apparatus of claim 12, wherein the fastening device includes hook-and-loop fasteners.

14. The apparatus of claim 13, wherein the fastening device includes a pair of first fastening patches provided on the mounting face, and a pair of second fastening patches provided to the one of the side walls of the lower leg support portion.

15. The apparatus of claim 14, wherein one of the pair of first fastening patches is arranged adjacent the first edge, and the other of the pair of first fastening patches is arranged adjacent the second edge.

16. The apparatus of claim 15, wherein the pair of first fastening patches are spaced apart at a distance from the one of the first fastening patches, the distance ranging between 1.5 inches and 4.5 inches.

17. The apparatus of claim 14, wherein each of the first fastening patches and the second fastening patches is selected from the group consisting of a hook patch and a loop patch.

18. A heel float therapeutic footwear apparatus comprising:
a heel float therapeutic boot including:
a lower leg support portion configured to support a lower leg of a patient, the lower leg support portion including a lower leg support cradle having a raised portion, the raised portion configured to elevate a heel of the patient when the patient is lying on the patient's back to reduce pressure on the patient's heel, the lower leg support portion having a front wall, a back wall, and opposite side walls, wherein the front wall at least partially surrounds a front portion of the lower leg, the back wall at least partially surrounds a back portion of the lower leg, and the side walls at least partially surrounds sides of the lower leg; and
a foot support portion connected to and extending from the lower leg support portion, the foot support portion including a foot support platform extending from the lower leg support cradle; and
a lateral support device detachably mounted to one of the side walls and configured to laterally support the lower leg support portion, the lateral support device having a mounting face abutted to the one of the side walls, the mounting face having a shape complementary to a shape of the side walls.

19. The heel float therapeutic footwear apparatus of claim 18, wherein the lateral support device is detachably mounted to the one of the side walls.

20. The heel float therapeutic footwear apparatus of claim 18, wherein the lateral support device has a first base face abutted to the mounting face at a first edge, and a second base face abutted to the mounting face at a second edge, the first base face abutted to the second base face at a third edge and arranged at an acute angle relative to the second base face.

21. The heel float therapeutic footwear apparatus according to claim 18, wherein the foot support platform includes:
an extended flap portion being configured to align with a base of the foot support portion, the extended flap portion having a proximal end bonded to a recess formed into a posterior flat surface of the lower leg support cradle adjacent the cutout region;
flaps extending from opposite sides of the extended flap portion; and
foam being positioned on the extended flap portion;
wherein the lower leg support cradle and the foot support platform together are connected at the recess.

22. The heel float therapeutic footwear apparatus of claim 18, further comprising:
a foot support structure having a first end portion and a second end portion, the foot support structure attached to the foot support platform at the first end portion, and attached at the second end portion to a side of the lower leg support portion, the foot support structure adjustably attached to the foot support platform and the side of the lower leg support portion to adjustably support a position and an angle of the foot support platform with respect to the lower leg support portion.

23. The heel float therapeutic footwear apparatus of claim 22, wherein the foot support structure removably attached to the foot support platform at the first end portion, and removably attached at the second end portion.

24. The heel float therapeutic footwear apparatus according to claim 22, wherein the foot support structure further comprises a left support wing and a right support wing attached on opposite sides of the boot, wherein the left support wing and the right support wing are spaced from each other.
